(19) United States
(12) Patent Application Publication
(10) Pub. No.: US 2021/0260069 A1
(43) Pub. Date: Aug. 26, 2021

(54) IRE1a INHIBITOR IN COMBINATION WITH CANCER THERAPEUTIC AGENT FOR CANCER TREATMENT

(71) Applicant: Fosun Orinove PharmaTech, Inc., Suzhou, Jiangsu (CN)

(72) Inventors: Qingping ZENG, Thousand Oaks, CA (US); John PATTERSON, Ventura, CA (US); Stephanie GREENE, Ventura, CA (US)

(21) Appl. No.: 17/261,413

(22) PCT Filed: Jul. 23, 2019
(86) PCT No.: PCT/CN2019/097291

§ 371 Date: Jan. 19, 2021

(30) Foreign Application Priority Data
Jul. 23, 2018 (CN) PCT/CN2018/096613
Nov. 2, 2018 (CN) PCT/CN2018/113783

(51) Int. Cl. A61K 31/5377; A61K 31/4412; A61K 45/06; A61P 35/04

(52) U.S. Cl. CPC A61K 31/5377; A61P 35/04; A61K 45/06; A61K 31/4412

(57) ABSTRACT

Provided are a pharmaceutical combination comprising an IRE1α inhibitor and one or more additional cancer therapeutic agents for the treatment of cancerous tumor, a pharmaceutical composition containing the same and a method for treating cancerous tumor using the same.

### TECHNICAL FIELD

Provided are a pharmaceutical combination comprising an IRE1α inhibitor and one or more additional cancer therapeutic agents for the treatment of cancerous tumor, a pharmaceutical composition containing the same and a method for treating cancerous tumor using the same.

### BACKGROUND

Cancer or cancerous tumor is the second leading cause of death in the developed world and is expected to kill more than 500,000 people in the United States this year. Despite sophisticated early detection techniques, new therapies and improved outcomes, new treatments are still required to improve patients' lives. One such area to this end is using combination therapies to target cancer from multiple weak points or multiple oncogenic drivers. Very often, cancer responds to treatments initially but the cancer reoccurs due to resistance and renewal of cancer stem cell survivors. Surgery, chemotherapy and radiotherapy, the traditional anti-cancer methods which may not result in complete responses or “cures”, can now be combined with targeted therapies and immunotherapies to improve patient survival outcomes versus using them as single agents.

The tumor microenvironment represents an underutilized therapeutic target area which impacts solid tumor growth and survival. Small molecule modulators of IRE-1α kinase and RNase functions have been reported with distinct mechanisms of action reflecting the engagement physically distinct binding sites and direct RNAse active site binding compounds represent a class of modulators that potently, reversibly, and selectively inhibit IRE-1α RNase activity including naphthalene (WO 2008/154484 A1; WO 2011/056744 A1) and coumarin (WO 2011/127070 A2) aromatic systems and which may be used as therapeutic agents to treat tumors.

### SUMMARY

In an aspect, provided is a pharmaceutical combination comprising

(a) a compound of formula (I) or a pharmaceutically acceptable salt thereof, and

(b) one or more additional cancer therapeutic agents,

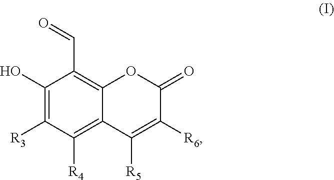

wherein

R₃ and R₄ are independently hydrogen or C₁₋₆ alkoxyl, which is optionally substituted with one or more substituents selected from the group consisting of (1) C₁-C₆ hydrocarbon chain containing 1 or 2 heteroatoms independently selected from the group consisting of N, O, and S, and (2) C₃₋₁₀ cycloalkyl, which optionally contains 1 or 2 heteroatoms independently selected from the group consisting of N, O, and S;

R₅ is hydrogen, C₁₋₆ alkyl, C₁₋₆ alkoxyl, or C₁₋₆ alkylamino;

R₆ is C₁₋₆ alkyl, which is substituted with 1, 2 or 3 substituents independently selected from the group consisting of C₁₋₆alkoxyl, C₁₋₆hydroxylalkyl, C₁₋₆alkoxyl C₁₋₆alkyl,

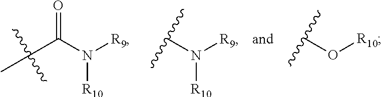

R₉ and R₁₀ are independently hydrogen; C₁₋₆ alkyl; C₁₋₆ alkoxyl C₁₋₆ alkyl; perfluoro C₁₋₆alkoxyl C₁₋₆alkyl; or

R₉ and R₁₀ together with the nitrogen atom to which they are attached form 3-10 membered heterocycle containing 1, 2, 3, or 4 heteroatoms independently selected from the group consisting of N, O, and S, and the heterocycle is optionally substituted with 1, 2, or 3 substituents independently selected from the group consisting of C₁₋₆ alkyl, C₁₋₆alkylamino, C₁₋₆ alkoxyl.

In an embodiment of the invention, the pharmaceutical combination is provided in the form of a pharmaceutical composition. In an alternative embodiment of the invention, the pharmaceutical combination is provided in the form of one or more kits.

In a further aspect, provided is a method for treating cancerous tumor, comprising administering a subject in need thereof an effective amount of the pharmaceutical combination according to the invention.

In a further aspect, provided is use of the pharmaceutical combination according to the invention for the manufacture of a medicament for treatment of cancerous tumor.

In a further aspect, provided is a method for treating cancerous tumor, comprising administering a subject in need thereof an effective amount of the compound of formula (I) or a pharmaceutically acceptable salt thereof, and one or more additional cancer therapeutic agents. In an alternative embodiment of the invention, the compound of formula (I) or a pharmaceutically acceptable salt thereof and the one or more additional cancer therapeutic agents are administered simultaneously, sequentially or separately.

In a yet further aspect, provided is a method for enhancing the efficacy of a cancer therapeutic agent comprising applying the compound of formula (I) or a pharmaceutically acceptable salt thereof in combination with the cancer therapeutic agent.

In a yet further aspect, provided is use of the compound of formula (I) or a pharmaceutically acceptable salt thereof for the manufacture of a medicament for treatment of cancerous tumor, wherein the medicament is for use in combination with one or more cancer therapeutic agents.

In a specific embodiment of the invention, the compound of formula (I) has the following formula (II) (which is also designated hereinafter as compound Orin 1001 or compound 4485):

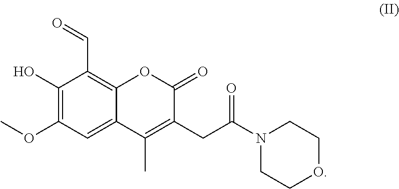 [SMILES: COc1cc2c(C)c(CC(=O)N3CCOCC3)c(=O)oc2c(C=O)c1O]

### BRIEF DESCRIPTION OF THE DRAWINGS

FIG. 1: Induction of percentage of XBP1s relative to total XBP1s and XBP1u measured by RT-qPCR to increasing concentrations of Lestaurtinib (X axis) in MM1s (circles), HEK293 (triangles), RPMI 8226 (diamonds) and H929 (squares) cells. Plots were generated using Excel fit software.

FIG. 2: Nilotinib induced greater than 50% XBP1s after 2 hours of treatment (triangles) but modest amounts at 1 (circles) or 4 hours (squares) of treatment of MM1s cells.

FIG. 3: Sorafenib induces highest levels of XBP1s after 1 (circles) or 2 (triangles) hours of treatment with modest levels after 4 hour (squares) of treatment of A549 cells.

FIG. 4: Dasatinib induces highest levels of XBP1s after 1 hour (circles) of treatment of A549 cells.

FIG. 5: Gefitinib induces highest levels of XBP1s after 2 hours of treatment (triangles) of A549 cells, increasing at 1 hour (circles).

FIG. 6: Indicated drugs induced potent XBP1s after 1 hour (circles), 2 hours (triangles) or 4 hours (squares) of treatment shown by IC₅₀ curves for Hepatoma (Hep G2, top panel), MCF-7 (mid panel) and RPMI 8226 cells (bottom panel).

FIG. 7: Torisel® induces high levels of XBP1s after 1 hour (circles) and 4 hours (squares) but modest levels after 2 hours of treatment (triangles) of A549 cells at indicated concentrations.

FIG. 8: Vorinostat induces high levels of XBP1s after 1 hour (circles) and little after 2 hours (triangles) or 4 hours (squares) of treatment of HT-29 cells at indicated concentrations.

FIG. 9: Paclitaxel induces high levels of XBP1s after 1 hour (circles) and with modest levels after 4 hours (squares) or 2 hours of treatment (triangles) of RPMI 8226 cells at indicated concentrations.

FIG. 10: Gemcitabine induces high levels of XBP1s after 4 hours (squares) but low levels after 1 hour (circles) or after 2 hours of treatment (triangles) of RPMI 8226 cells at indicated concentrations.

FIG. 11: 17-AAG induces high levels of XBP1s after 1 hour (circles) with modest levels after 2 hours (triangles) and low levels after 4 hours (squares) of treatment or of MCF-7 cells at indicated concentrations.

FIG. 12: 17-AAG induces high levels of XBP1s after 1 hour (circles) with modest levels after 2 hours (triangles) and low levels after 4 hours (squares) of treatment of Hepatoma cells at indicated concentrations.

FIG. 13: Intratumoral XBP-1 spliced effect of IRE-1 compound Orin 1001 in Velcade® treated RPMI xenografts.

FIGS. 14-21: Synergistic effects of compound Orin 1001 with other cancer therapeutic agents.

FIG. 22: Orin 1001 inhibits triple negative breast cancer in combination with eribulin, doxorubicin, cyclophosphamide, 5-FU or carboplatin in MDA-MB231-e551 xenograft model.

FIG. 23: XBP-1 splicing analysis of liver from compounds dosed PO.

### DETAILED DESCRIPTION

### Definition

Unless stated otherwise, the terms and phrases used herein have the following meaning. A specific term or phrase shall not be considered as unclear or indefinite when it is not specifically defined. It should be understood according to the general meaning in the art. The trade name used herein refers to the corresponding product or the active ingredient.

Unless specifically defined otherwise, proportion (including percentage) or part is calculated based on weight herein.

When used with a numerical variable, the term “approximate” or “about” usually refers to the value of the variable and all the values of the variable within the experimental error (for example, within an average 95% confidence interval) or within ±10% of the specified value, or a wider range.

As used herein, the singular forms “a,” “an” and “the” include plural referents unless the context clearly dictates otherwise.

The expression “comprise” or its synonyms “contain”, “include”, “have” or the like is open-ended, which does not exclude other unlisted elements, steps or ingredients. The expression “consist of” excludes any unlisted elements, steps or ingredients. The expression “substantially consist of” refers to specified elements, steps or ingredients within a given range, together with optional elements, steps or components which do not substantively affect the basic and novel feature of the claimed subject matter. It will be understood that the expression “comprise” encompasses the expressions “substantially consist of” and “consist of”.

The term “optional” or “optionally” means the event described subsequent thereto may or may not happen. This term encompasses the cases that the event may or may not happen.

The term “Cₘ₋ₙ” or “m-n membered” used herein means that the moiety has m-n carbon atoms or m-n atoms. For example, “C₁₋₆alkyl” means said alkyl has 1-6 carbon atoms. Likewise, C₃₋₁₀ cycloalkyl means said cycloalkyl has 3-10 carbon atoms. It will be understood that, when the term Cₘ₋ₙ is used in a group containing a moiety other than C-containing moiety, it refers to the carbon atom number in said C-containing moiety. For example, “C₁₋₆” in C₁₋₆hydroxylalkyl or C₁₋₆alkylamino means that the alkyl therein has 1-6 carbon atoms. In case more than one C-containing moieties are present, they are defined independently, for example C₁₋₆alkoxyl C₁₋₆alkyl. If only one Cₘ₋ₙ is defined, it should apply to all C-containing moieties, respectively, for example, C₁₋₆alkoxylalkyl means the alkoxyl and alkyl therein are each C₁₋₆ moiety.

It will be understood that the numerical range herein refers to each of the integers therein and any sub-range constituted by the integers. For example, “C₁₋₆” means said group may have 1 carbon atom, 2 carbon atoms, 3 carbon atoms, 4 carbon atoms, 5 carbon atoms or 6 carbon atoms. Accordingly, “C₁₋₆alkyl” encompasses “C₂₋₅alkyl”, “C₁₋₅alkyl”, “C₂₋₆alkyl” as well as C₁alkyl, C₂alkyl, C₃alkyl, C₄alkyl, C₅alkyl, C₆alkyl or the like.

The term “substitution” means one or more hydrogen atoms on a given atom are replaced by substituent(s), provided that the valence of the given atom is normal and the compound after substitution is stable.

The expression “one or more” or “at least one” refers to one, two, three, four, five, six, seven, eight, nine or more.

When any variable (e.g. R) occurs at the structure of a compound over one time, it is defined independently at each case. Therefore, for example, if a group is substituted by 0-2 R, the group may be optionally substituted by at most two R and R has independent option at each case. Additionally, a combination of substituents and/or the variants thereof are allowed only if such a combination will result in a stable compound.

Unless stated otherwise, the term “hetero” means heteroatom or heteroatom radical (i.e. a radical containing heteroatom), i.e. the atoms beyond carbon and hydrogen atoms or the radical containing such atoms. Preferably, the heteroatom(s) is independently selected from the group consisting of O, N, S and the like. In an embodiment wherein two or more heteroatoms are involved, the two or more heteroatoms may be the same, or part or all of the two or more heteroatoms may be different.

The term “alkyl”, either used alone or in combination with other group(s), refers to a linear or branched saturated aliphatic hydrocarbyl group composed of carbon and hydrogen atoms. The “alkyl” may be C₁₋₆alkyl. Non-limiting examples of C₁₋₆alkyl comprise but not limited to methyl, ethyl, propyl, isopropyl, n-butyl, isobutyl, sec-butyl, tert-butyl, pentyl, hexyl or the like.

The term “alkoxyl”, either used alone or in combination with other group(s), refers to an “alkyl” which is connected to the rest of the molecule via “—O—”, wherein the “alkyl” is defined as above. The “alkoxyl” may be C₁₋₆alkoxyl. Non-limiting examples of C₁₋₆alkoxyl comprise but not limited to methoxyl, ethoxyl, propoxy or the like.

The term “cycloalkyl”, either used alone or in combination with other group(s), refers to saturated monocyclic or polycyclic hydrocarbyl group composed of carbon and hydrogen atoms.

Cycloalkyl may contain 3-10, for example, 3-8, 3-7, 3-6, 3-5, 4-7, 4-6, or 3-4 carbon atoms or the like, or 3, 4, 5, 6, 7, 8, 9 or 10 carbon atoms. Non-limiting examples of C₃₋₁₀ cycloalkyl comprise but not limited to cyclopropyl, cyclobutyl, cyclopentyl, cyclohexyl or the like. The cycloalkyl may further optionally contain one or more (preferably 1 or 2) heteroatoms independently selected from the group consisting of N, O, and S. When one or more (preferably 1 or 2) heteroatoms are involved, the cycloalkyl is also known as heterocycloalkyl.

The term “heterocycle” or “heterocyclic”, either used alone or in combination with other group(s), refers to a saturated or unsaturated monocyclic or polycyclic system group, wherein part of the ring atoms (e.g. 1, 2, 3 or 4) are heteroatoms independently selected from the group consisting of N, O and S, and rest of the ring atoms are C. For example, 3-10 membered heterocycle contains 3-10 ring atoms in the system, wherein at least one ring atom (e.g. 1, 2, 3 or 4 preferably 1 or 2) is heteroatom selected from the group consisting of N, O and S. Preferably, heterocycle is 4-8 membered ring, more preferably 5-6 membered ring. Examples of 4 membered heterocycle comprise but not limited to azetidinyl. Examples of 5 membered heterocycle comprise but not limited to pyrrolidinyl, isoxazolidinyl, oxazolidinyl, isothiazolidinyl, thiazolidinyl, imidazolidinyl. Examples of 6 membered heterocycle comprise but not limited to piperidinyl, morpholinyl, piperazinyl. Examples of 7 membered heterocycle comprise but not limited to azacycloheptanyl, or the like.

The term “pharmaceutical combination” refers to a combined form of two or more active agents. It will be understood that these agents may be in a mixed or integrated form, e.g. a composition or mixture; or in a separated form, for example in separated compartments of a kit or in different kits. For example, the agents in the pharmaceutical combination may be formulated into one pharmaceutical composition for simultaneous administration. Alternatively, each of the agents may be individually formulated into an independent pharmaceutical composition, which may be administered simultaneously, sequentially or separately. The agents in the pharmaceutical combination may be given in an administration schedule that is synchronous, serial, overlapping, alternating, parallel, or any other treatment schedule in which the various agents are administered as part of a single treatment regimen. The active ingredient is exemplified as one or more agents according to the invention, for example a compound of formula (I) or a pharmaceutically acceptable salt thereof or one or more additional cancer therapeutic agents as mentioned herein.

The term “pharmaceutical composition” refers to an active agent(s), which is optionally combined with one or more pharmaceutically acceptable components (for example, but not limited to carrier). The active ingredient is exemplified as one or more agents according to the invention, for example a compound of formula (I) or a pharmaceutically acceptable salt thereof or one or more additional cancer therapeutic agents. In addition to the active ingredients, the pharmaceutical composition may further comprise one or more pharmaceutically acceptable carriers. A person skilled in the art will understand a compound of formula (I) or a pharmaceutically acceptable salt thereof and one or more additional cancer therapeutic agents may be formulated in one pharmaceutical composition, which can be used for e.g. simultaneous administration. Alternatively, a compound of formula (I) or a pharmaceutically acceptable salt thereof and one or more additional cancer therapeutic agents may be formulated in various pharmaceutical compositions, which can be used for e.g. simultaneous, sequential or separate administration. A person skilled in the art will also understand, the pharmaceutical composition(s) may independently and optionally comprise one or more pharmaceutically acceptable carriers.

The term “pharmaceutically acceptable carrier” refers to those carriers which have no significant irritation and do not impair the bioactivity and property of the active compound. This term may also be understood as inert substance which is administered with active ingredient and is beneficial to the administration thereof. Non-limiting examples include but not limited to any of the following substances which may optionally be approved by Food and Drug Administration for use in human or animal: glidant, sweetening agent, diluent, preservative, dye/colorant, flavoring agent, surfactant, wetting agent, dispersant, disintegrant, suspending agent, stabilizing agent, isotonic agent, solvent or emulsifying agent.

The term “administration” or “administrating” refers to a method that enables a compound, composition or combination to be delivered to a desired site of biological action. Such methods comprise but not limited to oral, parenteral (including intravenous, subcutaneous, intraperitoneal, intramuscular, intravascular injection or infusion), local, rectal administration or the like.

As used herein, the term “effective amount” refers to the amount of a medicament or agent or combination which is sufficient to achieve the desired effect. The effective amount may be determined individually and depends on the age and general condition of the receptor as well as specific active substance. The effective amount in specific case can be determined by a person skilled in the art through conventional test. When two or more agents are used in combination, for example, in the form of the pharmaceutical combination as claimed, the effective amount also refers to those of each of the agents exerting synergic effect.

The term “active ingredient”, “therapeutic agent”, “active substance” or “active agent” refers to a chemical entity useful for treating or preventing target disorder, disease or condition. Unless stated otherwise, the agent(s) (e.g. the additional cancer therapeutic agent(s)) in the pharmaceutical combination are commercially available or can be easily synthesized or obtained according to conventional means in the art.

The term “pharmaceutically acceptable” refers to the compound, material, composition, combination and/or dosage form, which are within the scope of reliable medical judgment, suitable for contact with human and animal tissues, without over toxicity, irritation, allergic reaction or other problems or complications and has acceptable benefit/risk ratio.

The term “combined preparation” used herein is defined as especially a “kit of parts” in the sense that the combination partners can be dosed independently or by use of different fixed combinations with distinguished amounts of the combination partners, i.e., simultaneously or at different time points. The parts of the kit of parts can then, e.g., be administered simultaneously or at different time points and with equal or different time intervals for any part of the kit of parts.

The terms “cancer” and “cancerous tumor” have the same meaning herein, and include but not limited to solid tumors and blood cancers. Exemplary solid tumors include but not limited to tumors of the breast, glioblastoma, bone, prostate, lung, adrenal gland (e.g., adrenocortical tumors), bile duct, bladder, bronchus, nervous tissue (including neuronal and glial tumors), gall bladder, stomach, salivary gland, esophagus, small intestine, cervix, colon, rectum, liver, ovary, pancreas, pituitary adenomas, and secretory adenomas. Exemplary blood cancers include but not limited to lymphomas and leukemia. Exemplary lymphomas include but not limited to multiple myeloma, Hodgkin's lymphoma, non-Hodgkin's lymphomas (e.g., cutaneous T cell lymphomas such as Sezary syndrome and Mycosis fungoides, diffuse large cell lymphoma, HTLV-1 associated T cell lymphoma, nodal peripheral T cell lymphoma, extranodal peripheral T cell lymphoma, central nervous system lymphoma, and AIDS-related lymphoma). Exemplary leukemia include but not limited to acute and chronic types of both lymphocytic and myelogenous leukemia (e.g. acute lymphocytic or lymphoblastic leukemia, acute myelogenous leukemia, acute myeloid leukemia, chronic myelogenous leukemia, chronic lymphocytic leukemia, T cell prolymphocyte leukemia, adult T cell leukemia, and hairy cell leukemia). In a particularly preferable embodiment, the “cancerous tumor” comprises triple negative breast cancer, estrogen positive breast cancer, ovarian carcinoma, pancreatic cancer, head and neck cancer, non-small cell lung cancer, glioblastoma, esophagus cancer, prostate cancer or multiple myeloma.

The “beneficial effect” herein for example refers to achieve additional advantageous therapeutic effects, diminish the incidence of side-effects or toxic effects (e.g., diarrhea or nausea), delay or slow down progression of cancer, reduce the tumor volume in a cancer patient, prolong survival of a cancer patient, prevent or delay tumor metastasis, decrease mortality and morbidity; or to sensitize a cancer patient to the cancer therapeutic agent(s) when an IRE-1α inhibitor is combined with the cancer therapeutic agent(s); or to reduce resistance to the cancer therapeutic agent(s) in a cancer patient who has been primarily resistant to such cancer therapeutic agent(s). In an embodiment, the beneficial effect refers to exerting synergic effect as compared with either of combination partners employed alone.

The term “subject” or “patient” used herein refers to mammal subject or patient, preferably human subject or patient.

### Pharmaceutical Combination

The present inventor surprisingly found that at least one beneficial effect for treating cancer is observed when the IRE1α inhibitor as a compound of formula (I) or a pharmaceutically acceptable salt thereof is employed in combination therapy, for example with one or more additional cancer therapeutic agents as recited herein.

Accordingly, in an aspect of the invention, provided is a pharmaceutical combination comprising:

(a) a compound of formula (I) or a pharmaceutically acceptable salt thereof

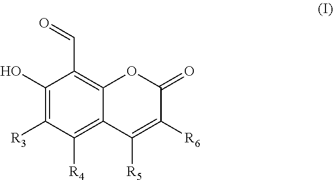

wherein

R₃ and R₄ are independently hydrogen or C₁₋₆ alkoxyl, which is optionally substituted with one or more substituents selected from the group consisting of (1) C₁-C₆ hydrocarbon chain containing 1 or 2 heteroatoms independently selected from the group consisting of N, O, and S, and (2) C₃₋₁₀ cycloalkyl, which optionally contains 1 or 2 heteroatoms independently selected from the group consisting of N, O, and S;

R₅ is hydrogen, C₁₋₆ alkyl, C₁₋₆ alkoxyl, or C₁₋₆ alkylamino;

R₆ is C₁₋₆ alkyl, which is substituted with 1, 2 or 3 substituents independently selected from the group consisting of C₁₋₆alkoxyl, C₁₋₆hydroxylalkyl, C₁₋₆alkoxyl C₁₋₆alkyl,

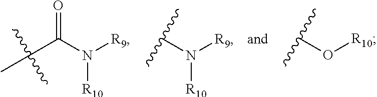

R₉ and R₁₀ are independently hydrogen; C₁₋₆ alkyl; C₁₋₆ alkoxyl C₁₋₆ alkyl; perfluoro C₁₋₆alkoxyl C₁₋₆alkyl; or

R₉ and R₁₀ together with the nitrogen atom to which they are attached form 3-10 membered heterocycle containing 1, 2, 3, or 4 heteroatoms independently selected from the group consisting of N, O, and S, and the heterocycle is optionally substituted with 1, 2, or 3 substituents independently selected from the group consisting of C₁₋₆ alkyl, C₁₋₆alkylamino, C₁₋₆ alkoxyl; and

(b) one or more additional cancer therapeutic agents.

According to a preferable embodiment, in the compound of formula (I) herein, R₃ is C₁₋₆ alkoxyl. According to a preferable embodiment, in the compound of formula (I) herein, R₄ is H. According to a preferable embodiment, in the compound of formula (I) herein, R₅ is C₁₋₆ alkyl. According to a preferable embodiment, in the compound of formula (I) herein, R₆ is C₁₋₆ alkyl (particularly C₁ alkyl), which is substituted with

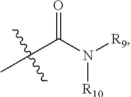

and R₉ and R₁₀ together with the nitrogen atom to which they are attached form a 6 membered heterocycle containing 1 or 2 heteroatoms independently selected from the group consisting of N and O (particularly morpholine).

In a specific embodiment of the invention, the compound of formula (I) has the following formula (II):

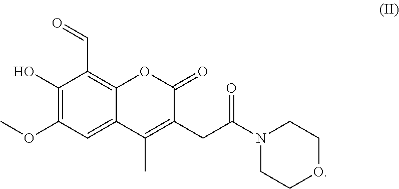 [SMILES: COc1cc2c(C)c(CC(=O)N3CCOCC3)c(=O)oc2c(C=O)c1O]

It will be understood that the additional cancer therapeutic agent(s) used in the combination according to the invention refers to a therapeutic agent(s) beyond the compound of formula (I) or pharmaceutically acceptable salt thereof as IRE1α inhibitor. Or in other words, the compound of formula (I) or pharmaceutically acceptable salt thereof is used as IRE1a inhibitor while the additional cancer therapeutic agent(s) is not an IRE1α inhibitor.

Inositol requiring enzyme-1α (IRE1a) is a transmembrane stress-sensing and signaling molecule that controls the Unfolded Protein Response (UPR). Numerous perturbations of protein folding contribute to Endoplasmic Reticulum (ER) stress. Downstream enzymatic activity is selectively activated during times of cellular stress, primarily during disease states and thus, inhibition of this pathway may impact tumor growth. Moreover, X-box protein 1 (XBP1) is activated in certain cancer types and may modulate the progression of disease. In vitro data shows that depletion of XBP1 inhibits tumor growth and relapse. XBP1 splicing activation is up-regulated in cancer and increased following chemotherapy and therefore, is suspected to play a key role in drug resistance.

The present inventor surprisingly found that various types of physiological stress induce the unfolded protein response including but not limited to hypoxia, nutrient starvation, acidosis, and genetic damage resulting in mutant or over-expressed misfolded proteins (oncogenic stress) and one or more of these conditions are manifest in cancer cells, which may in part be mediated by the microenvironment of the tumor. Without wishing to be bound to a theory, it is believed that the cytoprotective arm of the unfolded protein response (UPR) plays an anti-apoptotic role in tumor survival. In addition, bio- and chemotherapeutic drugs and radiation treatments may further impact the protein folding and degradation cycle in the ER thereby inducing the UPR as a protective resistance mechanism. Patients succumb to cancer because either the tumor is resistant to conventional therapies or returns in a resistant form after an initial response to treatment.

Although the compound of formula (I) or pharmaceutically acceptable salt thereof as IRE1α inhibitor per se can be used as cancer therapeutic agent, when it is used in combination with other cancer therapeutic agent, the efficacy for treating cancer can be enhanced. Chemotherapeutic agents, targeted small molecule oncology compounds, biomolecule etc. can directly induce ER stress and resulting UPR. IRE1α inhibitors can suppress this activation and thus can act synergistically for cellular proliferation inhibition when used in combination.

Accordingly, in an embodiment of the invention, the additional cancer therapeutic agent(s) has at least one of the following features:

(1) inducing ER stress;

(2) inducing or up-regulating IRE-1α expression;

(3) inducing or up-regulating XBP1 splicing; and

(4) being less effective when IRE-1α is expressed.

In an embodiment according to the invention, the one or more additional cancer therapeutic agents are selected from the group consisting of: cytotoxic chemotherapeutic agents; antimetabolites; antimitotic agents; alkylating agents; DNA damaging agents; antitumor antibiotics; platinum coordination complexes; proteasome inhibitors; HSP90 inhibitors; hormones and hormone analogs; aromatase inhibitors; fibrinolytic agents; antimigratory agents; antisecretory agents, e.g. brefeldin; immunosuppressives; anti-angiogenic compounds and vascular endothelial growth factor (VEGF) inhibitors; fibroblast growth factor (FGF/FGFR) inhibitors; epidermal growth factor receptor (EGFR) inhibitors; antibodies; checkpoint inhibitors; cell cycle inhibitors and differentiation inducers; mTOR inhibitors; corticosteroids; growth factor signal transduction kinase inhibitors; mitochondrial dysfunction inducers; caspase activators; chromatin disruptors and DNA repair enzyme inhibitors; HDAC inhibitors; Bcr-Abl inhibitors; FMS-like tyrosine kinase 3 (Flt3) inhibitors or any combination thereof.

Some non-limiting examples of the (one or more additional) cancer therapeutic agents are as follows:

1) cytotoxic chemotherapeutic agents, including microtubule disruptors such as taxane (e.g. paclitaxel, docetaxel, cabazitaxel, albumin-bound paclitaxel), eribulin, vincristin, vinblastin, nocodazole, epothilones and navelbine, and epipodophyllotoxins (e.g., teniposide);

2) antimetabolites such as pyrimidine analogs (e.g., 5-fluorouracil, floxuridine, capecitabine, gemcitabine and cytarabine), purine analogs, folate antagonists and related inhibitors (e.g., mercaptopurine, thioguanine, pentostatin and 2-chlorodeoxyadenosine), and folic acid analogs (e.g., methotrexate);

3) antimitotic agents such as vinca alkaloids (e.g., eribulin, vinblastine, vincristine, and vinorelbine);

4) alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC), and temozolomide;

5) DNA damaging agents, such as amsacrine, busulfan, camptothecin, irinotecan (CPT-11), topotecan, chlorambucil, cyclophosphamide, cytoxan, hexamethylmelamineoxaliplatin, iphosphamide, merchlorethamine, mitomycin, mitoxantrone, nitrosourea, plicamycin, procarbazine, teniposide, triethylenethiophosphoramide and etoposide (VP 16);

6) antitumor antibiotics, such as actinomycin, dactinomycin (actinomycin D), daunorubicin, doxorubicin (adriamycin), epirubicin, idarubicin, anthracyclines, mitoxantrone, bleomycins, plicamycin (mithramycin) and mitomycin;

7) platinum coordination complexes such as cisplatin, carboplatin, and oxaliplatin;

8) proteasome inhibitors, including bortezomib ([(1R)-3-methyl-1-[[(2S)-1-oxo-3-phenyl-2-[(pyrazinylcarbonyl)amino]propyl]amino]butyl] boronic acid; MG-341; VELCADE®), MG-132 (N-[(phenylmethoxy)carbonyl]-L-leucyl-N-[(1S)-1-formyl-3-methylbutyl]-L-leucinamide), carfilzomib (Kyprolis®) and ixazomib (Ninlaro®);

9) HSP90 inhibitors, including geldanamycin, radicicol, 17AAG, and Gamitrinib;

10) hormones and hormone analogs, including estrogen, goserelin, estrogen receptor inhibitors (e.g. raloxifene, tamoxifen, bazedoxifene), androgen receptor inhibitors (e.g. bicalutamide, nilutamide, enzalutamide), and androgen biosynthesis enzyme inhibitors (e.g. abiraterone);

11) aromatase inhibitors, e.g. letrozole, anastrozole; 12) fibrinolytic agents (such as tissue plasminogen activator, streptokinase and urokinase), including aspirin, COX-2 inhibitors, dipyridamole, ticlopidine, clopidogrel, abciximab;

13) antimigratory agents, e.g. somatostatin, wortmannin and PD98059;

14) antisecretory agents, e.g. brefeldin;

15) immunosuppressives, including cyclosporine, tacrolimus (FK-506), sirolimus (rapamycin), azathioprine, mycophenolate mofetil;

16) anti-angiogenic compounds (e.g., TNP 470, genistein) and vascular endothelial growth factor (VEGF) inhibitors such as ZD6474, sunitinib, vatalanib, sorafenib, bevacizumab;

17) fibroblast growth factor (FGF/FGFR) inhibitors such as BGJ398, AZD4547, dovitinib, lenvatinib, JNJ-42756493, GP369, BAY1187982;

18) epidermal growth factor receptor (EGFR) inhibitors such as afatinib, gefitinib, erlotinib;

19) antibodies, including trastuzumab (HERCEPTIN®), bevacizumab (AVASTIN®), cetuximab (ERBITUX®), rituximab (RITUXAN®);

20) checkpoint inhibitors, including CTLA4 inhibitors;

21) cell cycle inhibitors and differentiation inducers, e.g. tretinoin, ribociclib, palbociclib;

22) mTOR inhibitors, including rapamycin, everolimus, sirolimus, temsirolimus, ridaforolimus;

23) corticosteroids, including cortisone, dexamethasone, hydrocortisone, methylpednisolone, prednisone, and prenisolone; 24) growth factor signal transduction kinase inhibitors such as imatinib, erlotinib, sorafenib, sunitinib, lapatinib, trametinib, temozolomide;

25) mitochondrial dysfunction inducers such as α-tocopherol, Bcl-2 and Bcl-XL inhibitors such as venetoclax, ABT-737, navitoclax, obatoclax mesylate;

26) caspase activators such as 25-hydroxycholesterol, mitomycin C, proscillaridin A, zearalenone, fumonisin B1, garcinol;

27) chromatin disruptors and DNA repair enzyme inhibitors including PARP inhibitors such as 3-aminobenzamide, olaparib, talazoparib, niraparib, veliparib, rucaparib;

28) HDAC inhibitors, e.g. 17-AAG (TANESPIMYCIN®), suberoylanilide hydroxamic acid (SAHA®);

29) Bcr-Abl inhibitors, including imatinib, nilotinib, dasatinib, bosutinib, ponatinib;

30) FMS-like tyrosine kinase 3 (Flt3) inhibitors, including gilteritinib, lestaurtinib, midostaurin, nintedanib.

In a preferable embodiment, the additional cancer therapeutic agent is selected from the group consisting of:

Bcr-Abl inhibitors, such as imatinib, nilotinib, dasatinib, bosutinib, ponatinib, particularly nilotinib, dasatinib;

FMS-like tyrosine kinase 3 (Flt3) inhibitors, such as gilteritinib, lestaurtinib, midostaurin, nintedanib, particularly lestaurtinib;

anti-angiogenic compounds and vascular endothelial growth factor (VEGF) inhibitors, such as TNP 470, genistein, ZD6474, sunitinib, vatalanib, sorafenib, bevacizumab, vatalanib; particularly sorafenib, vatalinib;

epidermal growth factor receptor (EGFR) inhibitors, such as afatinib, gefitinib, erlotinib, particularly gefitinib;

mTOR inhibitors, such as apamycin, everolimus, sirolimus, temsirolimus, ridaforolimus, particularly temsirolimus;

HDAC inhibitors, such as 17-AAG (TANESPIMYCIN®), vorinostat (SAHA®), particularly vorinostat;

cytotoxic chemotherapeutic agents, such as microtubule disruptors such as taxane (e.g. paclitaxel, docetaxel, cabazitaxel, albumin-bound paclitaxel), eribulin, vincristin, vinblastin, nocodazole, epothilones and navelbine, and epipodophyllotoxins (e.g., teniposide), particularly taxane (e.g. paclitaxel, docetaxel, cabazitaxel) and eribulin;

antimetabolites such as pyrimidine analogs (e.g., 5-fluorouracil, floxuridine, capecitabine, gemcitabine and cytarabine), purine analogs, folate antagonists and related inhibitors (e.g., mercaptopurine, thioguanine, pentostatin and 2-chlorodeoxyadenosine), and folic acid analogs (e.g., methotrexate), particularly gemcitabine and 5-fluorouracil;

proteasome inhibitors, such as bortezomib, MG-132, carfilzomib, ixazomib, particularly bortezomib;

hormones and hormone analogs, including estrogen, goserelin, estrogen receptor inhibitors (e.g. raloxifene, tamoxifen, bazedoxifene), androgen receptor inhibitors (e.g. bicalutamide, nilutamide, enzalutamide), androgen biosynthesis enzyme inhibitors (e.g. abiraterone), particularly tamoxifen, enzalutamide and abiraterone;

alkylating agents, including nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC), and temozolomide, particularly cyclophosphamide and temozolomide;

antitumor antibiotics such as actinomycin, dactinomycin (actinomycin D), daunorubicin, doxorubicin (adriamycin), epirubicin, idarubicin, anthracyclines, mitoxantrone, bleomycins, plicamycin (mithramycin) and mitomycin, particularly doxorubicin;

platinum coordination complexes such as cisplatin, carboplatin, and oxaliplatin, particularly carboplatin;

aromatase inhibitors such as letrozole and anastrozole, particularly letrozole.

In a preferable embodiment, the additional cancer therapeutic agent is selected from the group consisting of cytotoxic chemotherapeutic agent, proteasome inhibitor, hormone analogue, alkylating agent, platinum coordination complex, antimetabolite, antitumor antibiotic, aromatase inhibitor, VEGF inhibitor or any combination thereof. More preferably, the cytotoxic chemotherapeutic agent is selected from the group consisting of microtubule disruptors, e.g. taxane or eribulin, and the taxane is selected from paclitaxel, docetaxel or cabazitaxel. More preferably, the proteasome inhibitor is bortezomib. More preferably, the hormone analogue is anti-estrogen agent, e.g. tamoxifen, androgen receptor inhibitor, e.g. enzalutamide, or androgen biosynthesis enzyme inhibitor, e.g. abiraterone. More preferably, the alkylating agent is cyclophosphamide or temozolomide. More preferably, the platinum coordination complex is carboplatin. More preferably, the antimetabolite is gemcitabine or 5-fluorouracil. More preferably, the antitumor antibiotic is doxorubicin. More preferably, the aromatase inhibitor is letrozole. More preferably, the VEGF inhibitor is sorafenib.

With respect to the pharmaceutical combination according to the invention, comprising a compound of formula (I) (for example, formula II) or a pharmaceutically acceptable salt thereof and (one or more) additional cancer therapeutic agents, the following embodiments are encompassed and exemplified.
The additional cancer therapeutic agent is sorafenib. The pharmaceutical combination is, for example, useful for treating liver tumor, particularly hepatocellular carcinoma.
The additional cancer therapeutic agent is a microtubule disruptor, wherein said microtubule disruptor is taxane or eribulin, and said taxane is selected from paclitaxel, docetaxel, cabazitaxel and albumin-bound paclitaxel, preferably paclitaxel and docetaxel, more preferably paclitaxel. The pharmaceutical combination is, for example, useful for treating breast cancer, particularly triple negative breast cancer.
The additional cancer therapeutic agent is cyclophosphamide, 5-fluorouracil, carboplatin or doxorubicin; preferably doxorubicin or carboplatin. The pharmaceutical combination is, for example, useful for treating breast cancer, particularly triple negative breast cancer.
The additional cancer therapeutic agent is taxane, wherein said taxane is selected from paclitaxel, docetaxel, cabazitaxel and albumin-bound paclitaxel, preferably paclitaxel and docetaxel, more preferably paclitaxel. The pharmaceutical combination is, for example, useful for treating breast cancer, particularly estrogen positive breast cancer, more particularly Her2 negative and estrogen positive metastatic breast cancer.
The additional cancer therapeutic agent is aromatase inhibitor, wherein said aromatase inhibitor is selected from letrozole and anastrozole. The pharmaceutical combination is, for example, useful for treating breast cancer, particularly estrogen positive breast cancer, more particularly Her2 negative and estrogen positive metastatic breast cancer.
The additional cancer therapeutic agent is tamoxifen. The pharmaceutical combination is, for example, useful for treating breast cancer, particularly estrogen positive breast cancer, more particularly Her2 negative and estrogen positive metastatic breast cancer.
The additional cancer therapeutic agent is taxane, wherein said taxane is selected from paclitaxel, docetaxel, cabazitaxel and albumin-bound paclitaxel, preferably paclitaxel and docetaxel, more preferably paclitaxel. The pharmaceutical combination is, for example, useful for treating esophagus cancer, particularly esophageal squamous cell cancer.
The additional cancer therapeutic agent is taxane, wherein said taxane is selected from paclitaxel, docetaxel, cabazitaxel and albumin-bound paclitaxel, preferably paclitaxel and docetaxel, more preferably paclitaxel. The pharmaceutical combination is, for example, useful for treating ovarian cancer.
The additional cancer therapeutic agent is doxorubicin. The pharmaceutical combination is, for example, useful for treating ovarian cancer.
The additional cancer therapeutic agent is taxane, wherein said taxane is selected from paclitaxel, docetaxel, cabazitaxel and albumin-bound paclitaxel, preferably paclitaxel and docetaxel, more preferably paclitaxel. The pharmaceutical combination is, for example, useful for treating lung cancer, particularly non-small cell lung cancer.
The additional cancer therapeutic agent is taxane, wherein said taxane is selected from paclitaxel, docetaxel, cabazitaxel and albumin-bound paclitaxel, preferably paclitaxel and docetaxel, more preferably paclitaxel. The pharmaceutical combination is, for example, useful for treating glioblastoma.
The additional cancer therapeutic agent is taxane, wherein said taxane is selected from paclitaxel, docetaxel, cabazitaxel and albumin-bound paclitaxel, preferably cabazitaxel. The pharmaceutical combination is, for example, useful for treating prostate cancer. The prostate cancer is, for example selected from androgen-dependent prostate cancer, hormone-refractory prostate cancer and castration-resistant prostate cancer, preferably said castration-resistant prostate cancer is metastatic.
The additional cancer therapeutic agent is abiraterone or enzalutamide. The pharmaceutical combination is, for example, useful for treating prostate cancer. The prostate cancer is, for example selected from androgen-dependent prostate cancer, hormone-refractory prostate cancer and castration-resistant prostate cancer, preferably said castration-resistant prostate cancer is metastatic.
The additional cancer therapeutic agent is gemcitabine. The pharmaceutical combination is, for example, useful for treating pancreatic cancer.
The additional cancer therapeutic agent is temozolomide. The pharmaceutical combination is, for example, useful for treating glioblastoma multiforme (GBM). More preferably, said glioblastoma multiforme is metastatic, recurrent, refractory or advanced.

The embodiments mentioned above can be applied independently or in combination.

In preferable embodiments of the pharmaceutical combination of the invention mentioned herein, the compound of formula (I) has formula (II):

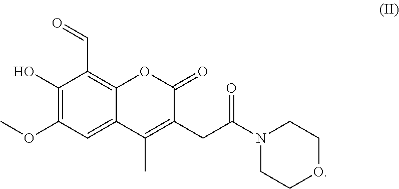 [SMILES: COc1cc2c(C)c(CC(=O)N3CCOCC3)c(=O)oc2c(C=O)c1O]

Accordingly, in a preferable embodiment, provided is a pharmaceutical combination, comprising a compound of formula (II) or a pharmaceutically acceptable salt thereof and one cytotoxic chemotherapeutic agent. In a more preferable embodiment, provided is a pharmaceutical combination, comprising a compound of formula (II) or a pharmaceutically acceptable salt thereof and one microtubule disruptor. In an even more preferable embodiment, provided is a pharmaceutical combination, comprising a compound of formula (II) or a pharmaceutically acceptable salt thereof and one microtubule disruptor selected from a taxane or eribulin. In an even further more preferable embodiment, provided is a pharmaceutical combination, comprising a compound of formula (II) or a pharmaceutically acceptable salt thereof and one microtubule disruptor selected from paclitaxel or docetaxel, or selected from cabazitaxel or eribulin. In a most preferable embodiment, provided is a pharmaceutical combination, comprising a compound of formula (II) or a pharmaceutically acceptable salt thereof and paclitaxel. In a most preferable embodiment, provided is a pharmaceutical combination, comprising a compound of formula (II) or a pharmaceutically acceptable salt thereof and cabazitaxel. In a most preferable embodiment, provided is a pharmaceutical combination, comprising a compound of formula (II) or a pharmaceutically acceptable salt thereof and eribulin.

In a preferable embodiment, provided is a pharmaceutical combination, comprising a compound of formula (II) or a pharmaceutically acceptable salt thereof and a proteasome inhibitor. In a more preferable embodiment, provided is a pharmaceutical combination, comprising a compound of formula (II) or a pharmaceutically acceptable salt thereof and bortezomib.

In a preferable embodiment, provided is a pharmaceutical combination, comprising a compound of formula (II) or a pharmaceutically acceptable salt thereof and a hormone analogue. In a more preferable embodiment, provided is a pharmaceutical combination, comprising a compound of formula (II) or a pharmaceutically acceptable salt thereof and an estrogen receptor inhibitor. In a more preferable embodiment, provided is a pharmaceutical combination, comprising a compound of formula (II) or a pharmaceutically acceptable salt thereof and an androgen receptor inhibitor. In a more preferable embodiment, provided is a pharmaceutical combination, comprising a compound of formula (II) or a pharmaceutically acceptable salt thereof and an androgen biosynthesis enzyme inhibitor. In an even more preferable embodiment, provided is a pharmaceutical combination, comprising a compound of formula (II) or a pharmaceutically acceptable salt thereof and an estrogen receptor inhibitor selected from tamoxifen. In an even more preferable embodiment, provided is a pharmaceutical combination, comprising a compound of formula (II) or a pharmaceutically acceptable salt thereof and an androgen receptor inhibitor selected from enzalutamide. In an even more preferable embodiment, provided is a pharmaceutical combination, comprising a compound of formula (II) or a pharmaceutically acceptable salt thereof and an androgen biosynthesis enzyme inhibitor selected from abiraterone.

In a preferable embodiment, provided is a pharmaceutical combination, comprising a compound of formula (II) or a pharmaceutically acceptable salt thereof and a VEGF inhibitor. In a more preferable embodiment, provided is a pharmaceutical combination, comprising a compound of formula (II) or a pharmaceutically acceptable salt thereof and sorafenib.

In a preferable embodiment, provided is a pharmaceutical combination, comprising a compound of formula (II) or a pharmaceutically acceptable salt thereof and an antimetabolite. In a more preferable embodiment, provided is a pharmaceutical combination, comprising a compound of formula (II) or a pharmaceutically acceptable salt thereof and 5-fluorouracil. In a more preferable embodiment, provided is a pharmaceutical combination, comprising a compound of formula (II) or a pharmaceutically acceptable salt thereof and gemcitabine.

In a preferable embodiment, provided is a pharmaceutical combination, comprising a compound of formula (II) or a pharmaceutically acceptable salt thereof and a platinum coordination complex. In a more preferable embodiment, provided is a pharmaceutical combination, comprising a compound of formula (II) or a pharmaceutically acceptable salt thereof and carboplatin.

In a preferable embodiment, provided is a pharmaceutical combination, comprising a compound of formula (II) or a pharmaceutically acceptable salt thereof and an antitumor antibiotic. In a more preferable embodiment, provided is a pharmaceutical combination, comprising a compound of formula (II) or a pharmaceutically acceptable salt thereof and doxorubicin.

In a preferable embodiment, provided is a pharmaceutical combination, comprising a compound of formula (II) or a pharmaceutically acceptable salt thereof and an aromatase inhibitor. In a more preferable embodiment, provided is a pharmaceutical combination, comprising a compound of formula (II) or a pharmaceutically acceptable salt thereof and letrozole.

In a preferable embodiment, provided is a pharmaceutical combination, comprising a compound of formula (II) or a pharmaceutically acceptable salt thereof and an alkylating agent. In a more preferable embodiment, provided is a pharmaceutical combination, comprising a compound of formula (II) or a pharmaceutically acceptable salt thereof and temozolomide. In a more preferable embodiment, provided is a pharmaceutical combination, comprising a compound of formula (II) or a pharmaceutically acceptable salt thereof and cyclophosphamide.

The embodiments mentioned above can be applied independently or in combination.

It will be understood that the agents in the pharmaceutical combination according to the invention, either the compound of formula (I) or the additional cancer therapeutic agent(s), encompass their other forms like stereoisomers, salts, prodrugs as well as crystal modifications, e.g. solvates and polymorphs and such forms are within the scope of the present invention. Preferably, these forms are pharmaceutically acceptable.

The effective amounts or dosages of the compound of formula (I) or the pharmaceutically acceptable salt thereof and one or more additional cancer therapeutic agents employed in the pharmaceutical combination according to the invention may vary depending on the particular compound or agent(s) employed, the mode of administration, the condition being treated, and severity of the condition being treated etc. Thus, the dosage regimen is selected in accordance with a variety of factors including the route of administration, the renal and hepatic function of the subject or the like. A physician, clinician or veterinarian of ordinary skill can readily determine and prescribe the effective amount required to prevent, counter or arrest the progress of the condition. Typically, the effective dosage of the compound of formula (I) or the pharmaceutically acceptable salt thereof for daily use is about 10-2000 mg, preferably 50-1000 mg for a warm-blooded animal like human of about 70 kg bodyweight. The effective dosage of the one or more additional cancer therapeutic agents for daily use in a warm-blooded animal, including man, can be determined by a package insert when said agent is provided as a marketed drug. It might be also be possible that the effective dosage of the one or more additional cancer therapeutic agents is adjusted according to species, age, individual condition, mode of administration, the clinical picture in question, etc.

In a particular embodiment, the pharmaceutical combination of the invention comprises

1) the compound of formula (II) or the pharmaceutically acceptable salt thereof

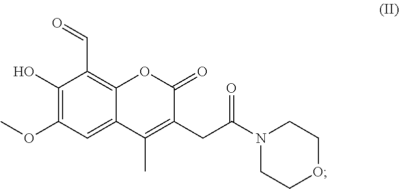 [SMILES: COc1cc2c(C)c(CC(=O)N3CCOCC3)c(=O)oc2c(C=O)c1O]

and

2) one or more additional cancer therapeutic agents.

In an embodiment of the invention, the additional cancer therapeutic agents are defined as above.

With respect to the pharmaceutical combination according to the invention, comprising a compound of formula (II) or a pharmaceutically acceptable salt thereof and (one or more) additional cancer therapeutic agents, the following embodiments regarding additional cancer therapeutic agent are encompassed and exemplified.
The additional cancer therapeutic agent is sorafenib. The pharmaceutical combination is, for example, useful for treating liver tumor, particularly hepatocellular carcinoma.
The additional cancer therapeutic agent is a microtubule disruptor, wherein said microtubule disruptor is taxane or eribulin, and said taxane is selected from paclitaxel, docetaxel, cabazitaxel and albumin-bound paclitaxel, preferably paclitaxel and docetaxel, more preferably paclitaxel. The pharmaceutical combination is, for example, useful for treating breast cancer, particularly triple negative breast cancer.
The additional cancer therapeutic agent is cyclophosphamide, 5-fluorouracil, carboplatin or doxorubicin; preferably doxorubicin or carboplatin. The pharmaceutical combination is, for example, useful for treating breast cancer, particularly triple negative breast cancer.
The additional cancer therapeutic agent is taxane, wherein said taxane is selected from paclitaxel, docetaxel, cabazitaxel and albumin-bound paclitaxel, preferably paclitaxel and docetaxel, more preferably paclitaxel. The pharmaceutical combination is, for example, useful for treating breast cancer, particularly estrogen positive breast cancer, more particularly Her2 negative and estrogen positive metastatic breast cancer.
The additional cancer therapeutic agent is aromatase inhibitor, wherein said aromatase inhibitor is selected from letrozole and anastrozole. The pharmaceutical combination is, for example, useful for treating breast cancer, particularly estrogen positive breast cancer, more particularly Her2 negative and estrogen positive metastatic breast cancer.
The additional cancer therapeutic agent is tamoxifen. The pharmaceutical combination is, for example, useful for treating breast cancer, particularly estrogen positive breast cancer, more particularly Her2 negative and estrogen positive metastatic breast cancer.
The additional cancer therapeutic agent is taxane, wherein said taxane is selected from paclitaxel, docetaxel, cabazitaxel and albumin-bound paclitaxel, preferably paclitaxel and docetaxel, more preferably paclitaxel. The pharmaceutical combination is, for example, useful for treating esophagus cancer, particularly esophageal squamous cell cancer.
The additional cancer therapeutic agent is taxane, wherein said taxane is selected from paclitaxel, docetaxel, cabazitaxel and albumin-bound paclitaxel, preferably paclitaxel and docetaxel, more preferably paclitaxel. The pharmaceutical combination is, for example, useful for treating ovarian cancer.
The additional cancer therapeutic agent is doxorubicin. The pharmaceutical combination is, for example, useful for treating ovarian cancer.
The additional cancer therapeutic agent is taxane, wherein said taxane is selected from paclitaxel, docetaxel, cabazitaxel and albumin-bound paclitaxel, preferably paclitaxel and docetaxel, more preferably paclitaxel. The pharmaceutical combination is, for example, useful for treating lung cancer, particularly non-small cell lung cancer.
The additional cancer therapeutic agent is taxane, wherein said taxane is selected from paclitaxel, docetaxel, cabazitaxel and albumin-bound paclitaxel, preferably paclitaxel and docetaxel, more preferably paclitaxel. The pharmaceutical combination is, for example, useful for treating glioblastoma.
The additional cancer therapeutic agent is taxane, wherein said taxane is selected from paclitaxel, docetaxel, cabazitaxel and albumin-bound paclitaxel, preferably cabazitaxel. The pharmaceutical combination is, for example, useful for treating prostate cancer. The prostate cancer is, for example selected from androgen-dependent prostate cancer, hormone-refractory prostate cancer and castration-resistant prostate cancer, preferably said castration-resistant prostate cancer is metastatic.
The additional cancer therapeutic agent is abiraterone or enzalutamide. The pharmaceutical combination is, for example, useful for treating prostate cancer. The prostate cancer is, for example selected from androgen-dependent prostate cancer, hormone-refractory prostate cancer and castration-resistant prostate cancer, preferably said castration-resistant prostate cancer is metastatic.
The additional cancer therapeutic agent is gemcitabine. The pharmaceutical combination is, for example, useful for treating pancreatic cancer.
The additional cancer therapeutic agent is temozolomide. The pharmaceutical combination is, for example, useful for treating glioblastoma multiforme (GBM). More preferably, said glioblastoma multiforme is metastatic, recurrent, refractory or advanced.

The embodiments mentioned above can be applied independently or in combination.

### Pharmaceutical Composition and Kits

The pharmaceutical combination according to the invention can further comprise one or more pharmaceutically acceptable carriers. In an embodiment wherein the pharmaceutical combination is provided in a unique form such as a pharmaceutical composition or mixture, the compound or agent(s) contained therein are combined with the same pharmaceutically acceptable carriers, for simultaneous, separate or sequential use. Accordingly, provided is a pharmaceutical composition, comprising the pharmaceutical combination according to the invention.

The pharmaceutical composition according to the invention can be prepared in a manner known per se and are those suitable for enteral, such as oral or rectal, and parenteral administration to mammals (warm-blooded animals), including man, comprising a therapeutically effective amount of compound of formula (I) and at least a therapeutically effective amount of cancer therapeutic agent, or further in combination with one or more pharmaceutically acceptable carries, especially suitable for enteral or parenteral application.

In an alternative embodiment wherein the pharmaceutical combination is provided in separated forms such as different compartments in a kit or in different kits, the agents contained therein, either the compound of formula (I) or the additional cancer therapeutic agent(s), are independently combined with the pharmaceutically acceptable carriers. The pharmaceutically acceptable carriers for each of the agent may be identical or different according to practice requirement. Accordingly, provided is also a kit, comprising (a) a compound of formula (I) or pharmaceutically acceptable salt thereof and optional one or more pharmaceutically acceptable carriers; (b) one or more additional cancer therapeutic agents and optional one or more pharmaceutically acceptable carriers; and (c) instruction for using (a) and (b). The compound of formula (I) or pharmaceutically acceptable salt thereof and the additional cancer therapeutic agents are defined as above. Accordingly, provided is a kit, comprising the pharmaceutical combination according to the invention.

The ratio of the total amounts of the compound of formula (I) or the pharmaceutically acceptable salt thereof to one or more additional cancer therapeutic agents in the pharmaceutical combination according to the invention can be varied, e.g. in order to cope with the needs of a patient sub-population to be treated or the needs of a single patient which different needs can be due to the particular disease, age, sex, body weight, etc.

### Methods and Use According to the Invention

In another aspect according to the invention, provided is a method for treating cancerous tumor, comprising administering a subject in need thereof an effective amount of the pharmaceutical combination according to the invention, wherein the active agents comprised in the pharmaceutical combination are defined as above.

Provided is also a method for treating cancerous tumor, comprising administering a subject in need thereof an effective amount of the pharmaceutical composition or kit according to the invention, wherein the pharmaceutical composition or kit comprises the pharmaceutical combination as defined above.

In an embodiment of the method of the invention, the pharmaceutical combination comprises

(a) a compound of formula (I) or a pharmaceutically acceptable salt thereof:

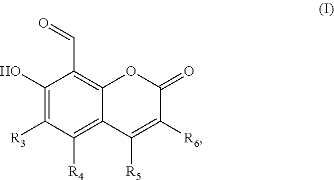

wherein R³, R⁴, R⁵ and R⁶ are defined as above; and

(b) one or more additional cancer therapeutic agents.

In a specific aspect of the invention, provided is a method for treating cancerous tumor, comprising administering a subject in need thereof an effective amount of a compound of formula (I) or a pharmaceutically acceptable salt thereof

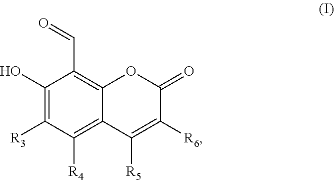

wherein R³, R⁴, R⁵ and R⁶ are defined as above; and

one or more additional cancer therapeutic agents.

In an embodiment of the methods above, the one or more additional cancer therapeutic agents are defined as above. In a specific embodiment, the cancer therapeutic agent has at least one of the following features: (1) inducing ER stress; (2) inducing or up-regulating IRE-1α expression; (3) inducing or up-regulating XBP1 splicing; and (4) being less effective when IRE-1α is expressed.

In some embodiments, said treatment is to cure the disease or to have an effect on disease regression or on the delay of progression of the disease. In an embodiment, said treatment is to inhibit the growth of tumor, for example, to reduce tumor volume, to delay the growth of tumor, to reverse the growth of tumor or any combination thereof. In another embodiment, said treatment is to kill the tumor, for example, to maintain the growth under a very low level.

Upon administration of the pharmaceutical combination according to the invention, the agents comprised therein (either compound of formula (I) or pharmaceutically acceptable salt thereof or one or more additional cancer therapeutic agents) are intended for simultaneous, separate or sequential use. Alternatively, upon administration of compound of formula (I) or pharmaceutically acceptable salt thereof and one or more additional cancer therapeutic agents, such agents are intended for simultaneous, separate or sequential use.

For example, the compound of formula (I) or pharmaceutically acceptable salt thereof and one or more additional cancer therapeutic agents can be used, e.g. as a combined preparation or a pharmaceutical composition/mixture such that they can be administered at essentially the same time. Alternatively, the compound of formula (I) or a pharmaceutically acceptable salt thereof and one or more additional cancer therapeutic agents can be in different compartments of a kit or different kits such that they can be administered at different time. For administration at different time, mention can be given to an order according to practical requirement. The compound of formula (I) or the pharmaceutically acceptable salt thereof can be administered before, after or along with the additional cancer therapeutic agent. The time interval between administrations of these agents may be several minutes, hours, days, months or even longer according to practical requirement.

Moreover, when the agents in the combination according to the invention are administered at different time points, the time intervals are such that the effect on the treated cancer in the combined use is larger than the effect which would be obtained by use of only any one of the combination partners.

If needed, the agents in the pharmaceutical combination according to the invention may be administered in the same or different routes. For example, the compound of formula (I) or pharmaceutically acceptable salt thereof and the additional cancer therapeutic agent(s) may be both administered orally or intravenously. Alternatively, the compound of formula (I) or pharmaceutically acceptable salt thereof may be administered orally while the additional cancer therapeutic agent(s) may be administered intravenously and vice versa. It would be understood that when more than one additional cancer therapeutic agents are used, their administration routes are selected independently, i.e. either identical or different.

In a specific embodiment according to the methods above, the compound of formula (I) has the following formula (II):

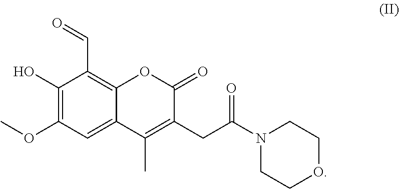 [SMILES: COc1cc2c(C)c(CC(=O)N3CCOCC3)c(=O)oc2c(C=O)c1O]

In yet a further aspect of the invention, provided is a method for enhancing the efficacy of a cancer therapeutic agent, comprising applying a compound of formula (I) or a pharmaceutically acceptable salt thereof in combination with the cancer therapeutic agent;

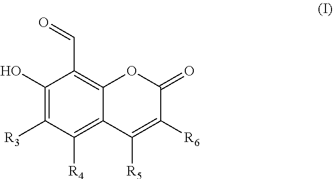

wherein, R³, R⁴, R⁵ and R⁶ are defined as above.

In a yet further aspect, provided is the use of the compound of formula (I) or a pharmaceutically acceptable salt thereof or a pharmaceutical combination comprising such agents for the manufacture of a medicament for treatment of cancerous tumor, wherein the medicament is for use in combination with one or more cancer therapeutic agents;

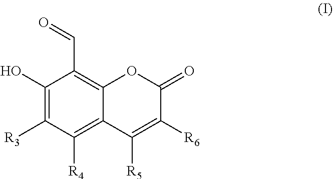

wherein, R³, R⁴, R⁵ and R⁶ are defined as above.

In a specific embodiment of the invention, the compound of formula (I) has the following formula (II):

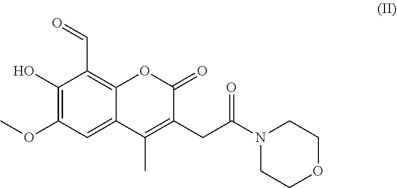 [SMILES: COc1cc2c(C)c(CC(=O)N3CCOCC3)c(=O)oc2c(C=O)c1O]

In an embodiment of the invention, the cancer therapeutic agent has at least one of the following features:

(1) inducing ER stress;

(2) inducing or up-regulating IRE-1α expression;

(3) inducing or up-regulating XBP1 splicing; and

(4) being less effective when IRE-1α is expressed.

In an embodiment of the invention, the enhancement of efficacy is embodied in inhibiting the growth of tumor, for example, reducing tumor volume, delaying the growth of tumor, reversing the growth of tumor or any combination thereof. Alternatively, enhancement of efficacy is embodied in killing the tumor, for example, maintaining the growth under a very low level.

In an embodiment of the invention, the one or more additional cancer therapeutic agents or the cancer therapeutic agent, of which the efficacy to be enhanced, or with which the compound of formula (I) is combined for use in manufacture of a medicament for treatment of cancerous tumor, are selected from the group consisting of cytotoxic chemotherapeutic agents; antimetabolites; antimitotic agents; alkylating agents; DNA damaging agents; antitumor antibiotics; platinum coordination complexes; proteasome inhibitors; HSP90 inhibitors; hormones and hormone analogs; aromatase inhibitors; fibrinolytic agents; antimigratory agents; antisecretory agents, e.g. brefeldin; immunosuppressives; anti-angiogenic compounds and vascular endothelial growth factor (VEGF) inhibitors; fibroblast growth factor (FGF/FGFR) inhibitors; epidermal growth factor receptor (EGFR) inhibitors; antibodies; checkpoint inhibitors; cell cycle inhibitors and differentiation inducers; mTOR inhibitors; corticosteroids; growth factor signal transduction kinase inhibitors; mitochondrial dysfunction inducers; caspase activators; chromatin disruptors and DNA repair enzyme inhibitors; HDAC inhibitors; Bcr-Abl inhibitors; FMS-like tyrosine kinase 3 (Flt3) inhibitors or any combination thereof.

Some examples of the cancer therapeutic agents are as follows:

1) cytotoxic chemotherapeutic agents, including microtubule disruptors such as taxane (e.g. paclitaxel, docetaxel, cabazitaxel, albumin-bound paclitaxel), eribulin, vincristin, vinblastin, nocodazole, epothilones and navelbine, and epipodophyllotoxins (e.g., teniposide);

2) antimetabolites such as pyrimidine analogs (e.g., 5-fluorouracil, floxuridine, capecitabine, gemcitabine and cytarabine), purine analogs, folate antagonists and related inhibitors (e.g., mercaptopurine, thioguanine, pentostatin and 2-chlorodeoxyadenosine), and folic acid analogs (e.g., methotrexate);

3) antimitotic agents such as vinca alkaloids (e.g., eribulin, vinblastine, vincristine, and vinorelbine);

4) alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC), and temozolomide;

5) DNA damaging agents such as amsacrine, busulfan, camptothecin, irinotecan (CPT-11), topotecan, chlorambucil, cyclophosphamide, cytoxan, hexamethylmelamineoxaliplatin, iphosphamide, merchlorethamine, mitomycin, mitoxantrone, nitrosourea, plicamycin, procarbazine, teniposide, triethylenethiophosphoramide and etoposide (VP 16);

6) antitumor antibiotics such as actinomycin, dactinomycin (actinomycin D), daunorubicin, doxorubicin (adriamycin), epirubicin, idarubicin, anthracyclines, mitoxantrone, bleomycins, plicamycin (mithramycin) and mitomycin;

7) platinum coordination complexes such as cisplatin, carboplatin, and oxaliplatin;

8) proteasome inhibitors, including bortezomib ([(1R)-3-methyl-1-[[(2S)-1-oxo-3-phenyl-2-[(pyrazinylcarbonyl)amino]propyl]amino]butyl] boronic acid; MG-341; VELCADE®), MG-132 (N-[(phenylmethoxy)carbonyl]-L-leucyl-N-[(1S)-1-formyl-3-methylbutyl]-L-leucinamide), carfilzomib (Kyprolis®) and ixazomib (Ninlaro®);

9) HSP90 inhibitors, including geldanamycin, radicicol, 17AAG, and gamitrinib;

10) hormones and hormone analogs, including estrogen, goserelin, estrogen receptor inhibitors (e.g. raloxifene, tamoxifen, bazedoxifene), androgen receptor inhibitors (e.g. bicalutamide, nilutamide, enzalutamide), and androgen biosynthesis enzyme inhibitors (e.g. abiraterone);

11) aromatase inhibitors, e.g. letrozole, anastrozole;

12) fibrinolytic agents (such as tissue plasminogen activator, streptokinase and urokinase), including aspirin, COX-2 inhibitors, dipyridamole, ticlopidine, clopidogrel, abciximab;

13) antimigratory agents, e.g. somatostatin, wortmannin and PD98059;

14) antisecretory agents, e.g. brefeldin;

15) immunosuppressives, including cyclosporine, tacrolimus (FK-506), sirolimus (rapamycin), azathioprine, mycophenolate mofetil;

16) anti-angiogenic compounds (e.g., TNP 470, genistein) and vascular endothelial growth factor (VEGF) inhibitors such as ZD6474, sunitinib, vatalanib, sorafenib, bevacizumab;

17) fibroblast growth factor receptor (FGF/FGFR) inhibitors such as BGJ398, AZD4547, dovitinib, lenvatinib, JNJ-42756493, GP369, BAY1187982;

18) epidermal growth factor (EGFR) inhibitors such as afatinib, gefitinib, erlotinib;

19) antibodies, including trastuzumab (HERCEPTIN®), bevacizumab (AVASTIN®), cetuximab (ERBITUX®), rituximab (RITUXAN®);

20) checkpoint inhibitors, including CTLA4 inhibitors,

21) cell cycle inhibitors and differentiation inducers, e.g. tretinoin, ribociclib, palbociclib;

22) mTOR inhibitors, including rapamycin, everolimus, sirolimus, temsirolimus, ridaforolimus;

23) corticosteroids, including cortisone, dexamethasone, hydrocortisone, methylpednisolone, prednisone, and prenisolone;

24) growth factor signal transduction kinase inhibitors such as imatinib, erlotinib, sorafenib, sunitinib, lapatinib, trametinib, temozolomide;

25) mitochondrial dysfunction inducers such as α-tocopherol, Bcl-2 and Bcl-XL inhibitors such as venetoclax, ABT-737, navitoclax, obatoclax mesylate;

26) caspase activators such as 25-hydroxycholesterol, mitomycin C, proscillaridin A, zearalenone, fumonisin B1, garcinol;

27) chromatin disruptors and DNA repair enzyme inhibitors including PARP inhibitors such as 3-aminobenzamide, olaparib, talazoparib, niraparib, veliparib, rucaparib;

28) HDAC inhibitors, e.g. 17-AAG (TANESPIMYCIN®), suberoylanilide hydroxamic acid (SAHA®);

29) Bcr-Abl inhibitors, including imatinib, nilotinib, dasatinib, bosutinib, ponatinib;

30) FMS-like tyrosine kinase 3 (Flt3) inhibitors, including gilteritinib, lestaurtinib, midostaurin, nintedanib.

In a preferable embodiment of the invention, the additional cancer therapeutic agent is selected from the group consisting of:

Bcr-Abl inhibitors, such as imatinib, nilotinib, dasatinib, bosutinib, ponatinib, particularly nilotinib, dasatinib;

FMS-like tyrosine kinase 3 (Flt3) inhibitors, such as gilteritinib, lestaurtinib, midostaurin, nintedanib, particularly lestaurtinib;

anti-angiogenic compounds and vascular endothelial growth factor (VEGF) inhibitors, such as TNP 470, genistein, ZD6474, sunitinib, vatalanib, sorafenib, bevacizumab, vatalanib; particularly sorafenib, vatalinib;

epidermal growth factor receptor (EGFR) inhibitors, such as afatinib, gefitinib, erlotinib, particularly gefitinib;

mTOR inhibitors, such as apamycin, everolimus, sirolimus, temsirolimus, ridaforolimus, particularly temsirolimus;

HDAC inhibitors, such as 17-AAG (TANESPIMYCIN®), vorinostat (SAHA®), particularly vorinostat;

cytotoxic chemotherapeutic agents, such as microtubule disruptors such as taxane (e.g. paclitaxel, docetaxel, cabazitaxel, albumin-bound paclitaxel), eribulin, vincristin, vinblastin, nocodazole, epothilones and navelbine, and epipodophyllotoxins (e.g., teniposide), particularly taxane (paclitaxel, docetaxel, cabazitaxel) and eribulin;

antimetabolites such as pyrimidine analogs (e.g., 5-fluorouracil, floxuridine, capecitabine, gemcitabine and cytarabine), purine analogs, folate antagonists and related inhibitors (e.g., mercaptopurine, thioguanine, pentostatin and 2-chlorodeoxyadenosine), and folic acid analogs (e.g., methotrexate), particularly gemcitabine and 5-fluorouracil;

proteasome inhibitors, such as bortezomib, MG-132, carfilzomib, ixazomib, particularly bortezomib;

hormones and hormone analogs, including estrogen, goserelin, estrogen receptor inhibitors (e.g. raloxifene, tamoxifen, bazedoxifene), androgen receptor inhibitors (e.g. bicalutamide, nilutamide, enzalutamide), androgen biosynthesis enzyme inhibitors (e.g. abiraterone), particularly tamoxifen, enzalutamide and abiraterone;

alkylating agents, including nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC), and temozolomide, particularly cyclophosphamide and temozolomide;

antitumor antibiotics such as actinomycin, dactinomycin (actinomycin D), daunorubicin, doxorubicin (adriamycin), epirubicin, idarubicin, anthracyclines, mitoxantrone, bleomycins, plicamycin (mithramycin) and mitomycin, particularly doxorubicin;

platinum coordination complexes such as cisplatin, carboplatin, and oxaliplatin, particularly carboplatin;

aromatase inhibitors such as letrozole and anastrozole, particularly letrozole.

In a preferable embodiment of the invention, the additional cancer therapeutic agent used in the method for treatment of cancerous tumor, or the cancer therapeutic agent of which the efficacy to be enhanced, or the cancer therapeutic agent with which the compound of formula (I) is combined for use in manufacture of a medicament for treatment of cancerous tumor, is selected from the group consisting of cytotoxic chemotherapeutic agent, proteasome inhibitor, hormone analogue, alkylating agent, platinum coordination complex, antimetabolite, antitumor antibiotic, aromatase inhibitor, VEGF inhibitor or any combination thereof. More preferably, the cytotoxic chemotherapeutic agent is selected from the group consisting of microtubule disruptors, e.g. taxane or eribulin, and the taxane is selected from paclitaxel, docetaxel or cabazitaxel. More preferably, the proteasome inhibitor is bortezomib. More preferably, the hormone analogue is anti-estrogen agent, e.g. tamoxifen, androgen receptor inhibitor, e.g. enzalutamide, or androgen biosynthesis enzyme inhibitor, e.g. abiraterone. More preferably, the alkylating agent is cyclophosphamide or temozolomide. More preferably, the platinum coordination complex is carboplatin. More preferably, the antimetabolite is gemcitabine or 5-fluorouracil. More preferably, the antitumor antibiotic is doxorubicin. More preferably, the aromatase inhibitor is letrozole.

More preferably, the VEGF inhibitor is sorafenib.

With respect to the methods according to the invention, the following embodiments are encompassed and exemplified.
A method for treating liver tumor, comprising administering a subject in need thereof an effective amount of a compound of formula (I) or a pharmaceutically acceptable salt thereof and sorafenib, or a pharmaceutical combination comprising such agents. The liver tumor is for example, hepatocellular carcinoma.
A method for treating breast cancer, comprising administering a subject in need thereof an effective amount of a compound of formula (I) or a pharmaceutically acceptable salt thereof and a microtubule disruptor, or a pharmaceutical combination comprising such agents, wherein said microtubule disruptor is selected from taxane and eribulin, and said taxane selected from paclitaxel, docetaxel, cabazitaxel and albumin-bound paclitaxel, preferably paclitaxel and docetaxel, more preferably paclitaxel. The breast cancer is for example triple negative breast cancer.
A method for treating breast cancer, comprising administering a subject in need thereof an effective amount of a compound of formula (I) or a pharmaceutically acceptable salt thereof and an agent selected from cyclophosphamide, 5-fluorouracil, carboplatin and doxorubicin; preferably doxorubicin or carboplatin, or a pharmaceutical combination comprising such agents. The breast cancer is for example triple negative breast cancer.
A method for treating breast cancer, comprising administering a subject in need thereof an effective amount of a compound of formula (I) or a pharmaceutically acceptable salt thereof and a taxane, or a pharmaceutical combination comprising such agents, wherein said taxane is selected from paclitaxel, docetaxel, cabazitaxel and albumin-bound paclitaxel, preferably paclitaxel and docetaxel, more preferably paclitaxel. The breast cancer is for example estrogen positive breast cancer, particularly Her2 negative and estrogen positive metastatic breast cancer.
A method for treating breast cancer, comprising administering a subject in need thereof an effective amount of a compound of formula (I) or a pharmaceutically acceptable salt thereof and a aromatase inhibitor, or a pharmaceutical combination comprising such agents, wherein said aromatase inhibitor is selected from letrozole and anastrozole. The breast cancer is for example estrogen positive breast cancer, particularly Her2 negative and estrogen positive metastatic breast cancer.
A method for treating breast cancer, comprising administering a subject in need thereof an effective amount of a compound of formula (I) or a pharmaceutically acceptable salt thereof and tamoxifen, or a pharmaceutical combination comprising such agents. The breast cancer is for example estrogen positive breast cancer, particularly Her2 negative and estrogen positive metastatic breast cancer.
A method for treating esophagus cancer, comprising administering a subject in need thereof an effective amount of a compound of formula (I) or a pharmaceutically acceptable salt thereof and a taxane, or a pharmaceutical combination comprising such agents, wherein said taxane is selected from paclitaxel, docetaxel, cabazitaxel and albumin-bound paclitaxel, preferably paclitaxel and docetaxel, more preferably paclitaxel. The esophagus cancer is for example, esophageal squamous cell cancer.
A method for treating ovarian cancer, comprising administering a subject in need thereof an effective amount of a compound of formula (I) or a pharmaceutically acceptable salt thereof and a taxane, or a pharmaceutical combination comprising such agents, wherein said taxane is selected from paclitaxel, docetaxel, cabazitaxel and albumin-bound paclitaxel, preferably paclitaxel and docetaxel, more preferably paclitaxel.
A method for treating ovarian cancer, comprising administering a subject in need thereof an effective amount of a compound of formula (I) or a pharmaceutically acceptable salt thereof and doxorubicin, or a pharmaceutical combination comprising such agents.
A method for treating lung cancer, comprising administering a subject in need thereof an effective amount of a compound of formula (I) or a pharmaceutically acceptable salt thereof and a taxane, or a pharmaceutical combination comprising such agents, wherein said taxane is selected from paclitaxel, docetaxel, cabazitaxel and albumin-bound paclitaxel, preferably paclitaxel and docetaxel, more preferably paclitaxel. The lung cancer is for example non-small cell lung cancer.
A method for treating glioblastoma, comprising administering a subject in need thereof an effective amount of a compound of formula (I) or a pharmaceutically acceptable salt thereof and a taxane, or a pharmaceutical combination comprising such agents, wherein said taxane is selected from paclitaxel, docetaxel, cabazitaxel and albumin-bound paclitaxel, preferably paclitaxel and docetaxel, more preferably paclitaxel.
A method for treating prostate cancer, comprising administering a subject in need thereof an effective amount of a compound of formula (I) or a pharmaceutically acceptable salt thereof and a taxane, or a pharmaceutical combination comprising such agents, wherein said taxane is selected from paclitaxel, docetaxel, cabazitaxel and albumin-bound paclitaxel, preferably cabazitaxel. The prostate cancer is, for example selected from androgen-dependent prostate cancer, hormone-refractory prostate cancer and castration-resistant prostate cancer, preferably said castration-resistant prostate cancer is metastatic.
A method for treating prostate cancer, comprising administering a subject in need thereof an effective amount of a compound of formula (I) or a pharmaceutically acceptable salt thereof and abiraterone or enzalutamide, or a pharmaceutical combination comprising such agents. The prostate cancer is for example selected from androgen-dependent prostate cancer, hormone-refractory prostate cancer and castration-resistant prostate cancer, preferably said castration-resistant prostate cancer is metastatic.
A method for treating pancreatic cancer, comprising administering a subject in need thereof an effective amount of a compound of formula (I) or a pharmaceutically acceptable salt thereof and gemcitabine, or a pharmaceutical combination comprising such agents.
A method for treating glioblastoma multiforme (GBM), comprising administering a subject in need thereof an effective amount of a compound of formula (I) or a pharmaceutically acceptable salt thereof and temozolomide, or a pharmaceutical combination comprising such agents. The glioblastoma multiforme is for example metastatic, recurrent, refractory or advanced.

The embodiments mentioned above can be applied independently or in combination.

As mentioned above, the compound of formula (I) may have formula (II).

In the methods for enhancing the efficacy of a cancer therapeutic agent by applying a compound of formula (I) or a pharmaceutically acceptable salt thereof in combination with the cancer therapeutic agent, some specific embodiments regarding the cancer therapeutic agents and cancers can be referred to the above and below embodiments of treating tumors.

In a preferable embodiment, provided is a method for treatment of cancerous tumor, comprising administering a subject in need thereof an effective amount of a compound of formula (II) or a pharmaceutically acceptable salt thereof and one cytotoxic chemotherapeutic agent. In a more preferable embodiment, provided is a method for treatment of cancerous tumor, comprising administering a subject in need thereof an effective amount of a compound of formula (II) or a pharmaceutically acceptable salt thereof and one microtubule disruptor. In an even more preferable embodiment, provided is a method for treatment of cancerous tumor, comprising administering a subject in need thereof an effective amount of a compound of formula (II) or a pharmaceutically acceptable salt thereof and a microtubule disruptor selected from taxane or eribulin. In an even further more preferable embodiment, provided is a method for treatment of cancerous tumor, comprising administering a subject in need thereof an effective amount of a compound of formula (II) or a pharmaceutically acceptable salt thereof and one microtubule disruptor selected from paclitaxel or docetaxel, or selected from cabazitaxel or eribulin. In a most preferable embodiment, provided is a method for treatment of cancerous tumor, comprising administering a subject in need thereof an effective amount of a compound of formula (II) or a pharmaceutically acceptable salt thereof and paclitaxel. In a most preferable embodiment, provided is a method for treatment of cancerous tumor, comprising administering a subject in need thereof an effective amount of a compound of formula (II) or a pharmaceutically acceptable salt thereof and cabazitaxel. In a most preferable embodiment, provided is a method for treatment of cancerous tumor, comprising administering a subject in need thereof an effective amount of a compound of formula (II) or a pharmaceutically acceptable salt thereof and eribulin.

In a preferable embodiment, provided is a method for treatment of cancerous tumor, comprising administering a subject in need thereof an effective amount of a compound of formula (II) or a pharmaceutically acceptable salt thereof and a proteasome inhibitor. In a more preferable embodiment, provided is a method for treatment of cancerous tumor, comprising administering a subject in need thereof an effective amount of a compound of formula (II) or a pharmaceutically acceptable salt thereof and bortezomib.

In a preferable embodiment, provided is a method for treatment of cancerous tumor, comprising administering a subject in need thereof an effective amount of a compound of formula (II) or a pharmaceutically acceptable salt thereof and a hormone analogue. In a more preferable embodiment, provided is a method for treatment of cancerous tumor, comprising administering a subject in need thereof an effective amount of a compound of formula (II) or a pharmaceutically acceptable salt thereof and an estrogen receptor inhibitor. In a more preferable embodiment, provided is a method for treatment of cancerous tumor, comprising administering a subject in need thereof an effective amount of a compound of formula (II) or a pharmaceutically acceptable salt thereof and an androgen receptor inhibitor. In a more preferable embodiment, provided is a method for treatment of cancerous tumor, comprising administering a subject in need thereof an effective amount of a compound of formula (II) or a pharmaceutically acceptable salt thereof and an androgen biosynthesis enzyme inhibitor. In an even more preferable embodiment, provided is a method for treatment of cancerous tumor, comprising administering a subject in need thereof an effective amount of a compound of formula (II) or a pharmaceutically acceptable salt thereof and an estrogen receptor inhibitor selected from tamoxifen. In an even more preferable embodiment, provided is a method for treatment of cancerous tumor, comprising administering a subject in need thereof an effective amount of a compound of formula (II) or a pharmaceutically acceptable salt thereof and an androgen receptor inhibitor selected from enzalutamide. In an even more preferable embodiment, provided is a method for treatment of cancerous tumor, comprising administering a subject in need thereof an effective amount of a compound of formula (II) or a pharmaceutically acceptable salt thereof and an androgen biosynthesis enzyme inhibitor selected from abiraterone.

In a preferable embodiment, provided is a method for treatment of cancerous tumor, comprising administering a subject in need thereof an effective amount of a compound of formula (II) or a pharmaceutically acceptable salt thereof and a VEGF inhibitor. In a more preferable embodiment, provided is a method for treatment of cancerous tumor, comprising administering a subject in need thereof an effective amount of a compound of formula (II) or a pharmaceutically acceptable salt thereof and sorafenib.

In a preferable embodiment, provided is a method for treatment of cancerous tumor, comprising administering a subject in need thereof an effective amount of a compound of formula (II) or a pharmaceutically acceptable salt thereof and an antimetabolite. In a more preferable embodiment, provided is a method for treatment of cancerous tumor, comprising administering a subject in need thereof an effective amount of a compound of formula (II) or a pharmaceutically acceptable salt thereof and 5-fluorouracil. In a more preferable embodiment, provided is a method for treatment of cancerous tumor, comprising administering a subject in need thereof an effective amount of a compound of formula (II) or a pharmaceutically acceptable salt thereof and gemcitabine.

In a preferable embodiment, provided is a method for treatment of cancerous tumor, comprising administering a subject in need thereof an effective amount of a compound of formula (II) or a pharmaceutically acceptable salt thereof and a platinum coordination complex.

In a more preferable embodiment, provided is a method for treatment of cancerous tumor, comprising administering a subject in need thereof an effective amount of a compound of formula (II) or a pharmaceutically acceptable salt thereof and carboplatin.

In a preferable embodiment, provided is a method for treatment of cancerous tumor, comprising administering a subject in need thereof an effective amount of a compound of formula (II) or a pharmaceutically acceptable salt thereof and an antitumor antitibiotic. In a more preferable embodiment, provided is a method for treatment of cancerous tumor, comprising administering a subject in need thereof an effective amount of a compound of formula (II) or a pharmaceutically acceptable salt thereof and doxorubicin.

In a preferable embodiment, provided is a method for treatment of cancerous tumor, comprising administering a subject in need thereof an effective amount of a compound of formula (II) or a pharmaceutically acceptable salt thereof and an aromatase inhibitor. In a more preferable embodiment, provided is a method for treatment of cancerous tumor, comprising administering a subject in need thereof an effective amount of a compound of formula (II) or a pharmaceutically acceptable salt thereof and letrozole.

In a preferable embodiment, provided is a method for treatment of cancerous tumor, comprising administering a subject in need thereof an effective amount of a compound of formula (II) or a pharmaceutically acceptable salt thereof and an alkylating agent. In a more preferable embodiment, provided is a method for treatment of cancerous tumor, comprising administering a subject in need thereof an effective amount of a compound of formula (II) or a pharmaceutically acceptable salt thereof and temozolomide. In a more preferable embodiment, provided is a method for treatment of cancerous tumor, comprising administering a subject in need thereof an effective amount of a compound of formula (II) or a pharmaceutically acceptable salt thereof and cyclophosphamide.

The following embodiments are also are encompassed and exemplified.
A method for treating liver tumor, comprising administering a subject in need thereof an effective amount of a pharmaceutical combination comprising a compound of formula (II) or a pharmaceutically acceptable salt thereof and sorafenib. The liver tumor is for example, hepatocellular carcinoma.
A method for treating breast cancer, comprising administering a subject in need thereof an effective amount of a pharmaceutical combination comprising a compound of formula (II) or a pharmaceutically acceptable salt thereof and a microtubule disruptor, wherein said microtubule disruptor is selected from taxane and eribulin, and said taxane selected from paclitaxel, docetaxel, cabazitaxel and albumin-bound paclitaxel, preferably paclitaxel and docetaxel, more preferably paclitaxel. The breast cancer is for example triple negative breast cancer.
A method for treating breast cancer, comprising administering a subject in need thereof an effective amount of a pharmaceutical combination comprising a compound of formula (II) or a pharmaceutically acceptable salt thereof and an agent selected from cyclophosphamide, 5-fluorouracil, carboplatin and doxorubicin; preferably doxorubicin or carboplatin. The breast cancer is for example triple negative breast cancer.
A method for treating breast cancer, comprising administering a subject in need thereof an effective amount of a pharmaceutical combination comprising a compound of formula (II) or a pharmaceutically acceptable salt thereof and a taxane, wherein said taxane is selected from paclitaxel, docetaxel, cabazitaxel and albumin-bound paclitaxel, preferably paclitaxel and docetaxel, more preferably paclitaxel. The breast cancer is for example estrogen positive breast cancer, particularly Her2 negative and estrogen positive metastatic breast cancer.
A method for treating breast cancer, comprising administering a subject in need thereof an effective amount of a pharmaceutical combination comprising a compound of formula (II) or a pharmaceutically acceptable salt thereof and a aromatase inhibitor, wherein said aromatase inhibitor is selected from letrozole and anastrozole. The breast cancer is for example estrogen positive breast cancer, particularly Her2 negative and estrogen positive breast metastatic cancer.
A method for treating breast cancer, comprising administering a subject in need thereof an effective amount of a pharmaceutical combination comprising a compound of formula (II) or a pharmaceutically acceptable salt thereof and tamoxifen. The breast cancer is for example estrogen positive breast cancer, particularly Her2 negative and estrogen positive metastatic breast cancer.
A method for treating esophagus cancer, comprising administering a subject in need thereof an effective amount of a pharmaceutical combination comprising a compound of formula (II) or a pharmaceutically acceptable salt thereof and a taxane, wherein said taxane is selected from paclitaxel, docetaxel, cabazitaxel and albumin-bound paclitaxel, preferably paclitaxel and docetaxel, more preferably paclitaxel. The esophagus cancer is for example, esophageal squamous cell cancer.
A method for treating ovarian cancer, comprising administering a subject in need thereof an effective amount of a pharmaceutical combination comprising a compound of formula (II) or a pharmaceutically acceptable salt thereof and a taxane, wherein said taxane is selected from paclitaxel, docetaxel, cabazitaxel and albumin-bound paclitaxel, preferably paclitaxel and docetaxel, more preferably paclitaxel.
A method for treating ovarian cancer, comprising administering a subject in need thereof an effective amount of a pharmaceutical combination comprising a compound of formula (II) or a pharmaceutically acceptable salt thereof and doxorubicin.
A method for treating lung cancer, comprising administering a subject in need thereof an effective amount of a pharmaceutical combination comprising a compound of formula (II) or a pharmaceutically acceptable salt thereof and a taxane, wherein said taxane is selected from paclitaxel, docetaxel, cabazitaxel and albumin-bound paclitaxel, preferably paclitaxel and docetaxel, more preferably paclitaxel. The lung cancer is for example non-small cell lung cancer.
A method for treating glioblastoma, comprising administering a subject in need thereof an effective amount of a pharmaceutical combination comprising a compound of formula (II) or a pharmaceutically acceptable salt thereof and a taxane, wherein said taxane is selected from paclitaxel, docetaxel, cabazitaxel and albumin-bound paclitaxel, preferably paclitaxel and docetaxel, more preferably paclitaxel.
A method for treating prostate cancer, comprising administering a subject in need thereof an effective amount of a pharmaceutical combination comprising a compound of formula (II) or a pharmaceutically acceptable salt thereof and abiraterone. The prostate cancer is for example selected from androgen-dependent prostate cancer, hormone-refractory prostate cancer and castration-resistant prostate cancer, preferably said castration-resistant prostate cancer is metastatic.
A method for treating prostate cancer, comprising administering a subject in need thereof an effective amount of a pharmaceutical combination comprising a compound of formula (II) or a pharmaceutically acceptable salt thereof and a taxane, wherein said taxane is selected from paclitaxel, docetaxel, cabazitaxel and albumin-bound paclitaxel, preferably cabazitaxel. The prostate cancer is, for example selected from androgen-dependent prostate cancer, hormone-refractory prostate cancer and castration-resistant prostate cancer, preferably said castration-resistant prostate cancer is metastatic.
A method for treating pancreatic cancer, comprising administering a subject in need thereof an effective amount of a pharmaceutical combination comprising a compound of formula (II) or a pharmaceutically acceptable salt thereof and gemcitabine.
A method for treating glioblastoma multiforme (GBM), comprising administering a subject in need thereof an effective amount of a pharmaceutical combination comprising a compound of formula (II) or a pharmaceutically acceptable salt thereof and temozolomide. The glioblastoma multiforme is for example metastatic, recurrent, refractory or advanced.

The embodiments mentioned above can be applied independently or in combination. It will be understood that the administered agents may be present in the pharmaceutical combination according to the invention.

In yet a further aspect, provided is the pharmaceutical combination according to the invention for treating cancerous tumor.

In still a further aspect, provided is use of the pharmaceutical combination according to the invention for the manufacture of a medicament for treating cancerous tumor.

In an embodiment, the pharmaceutical combination comprises

(a) a compound of formula (I) or a pharmaceutically acceptable salt thereof:

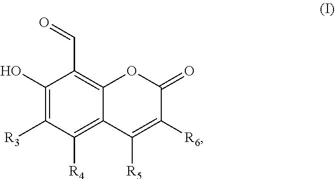

wherein R³, R⁴, R⁵ and R⁶ are defined as above; and

(b) one or more additional cancer therapeutic agents.

Preferably, the compound of formula (I) has the following formula (II):

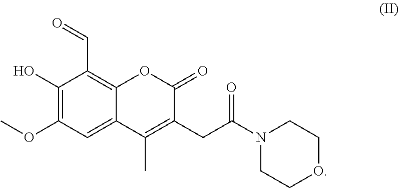 [SMILES: COc1cc2c(C)c(CC(=O)N3CCOCC3)c(=O)oc2c(C=O)c1O]

Preferably, the additional cancer therapeutic agents are defined as above. The cancerous tumors are also preferably defined as above.

### Cancerous Tumor

The cancerous tumors which can be treated with the pharmaceutical combination according to the invention or to which the efficacy of the cancer therapeutic agent can be enhanced comprise but not limited to solid tumors and blood cancers.

Exemplary solid tumors include, but are not limited to tumors of breast, glioblastoma, bone, prostate, lung, adrenal gland (e.g., adrenocortical tumors), bile duct, bladder, bronchus, nervous tissue (including neuronal and glial tumors), gall bladder, stomach, salivary gland, esophagus, small intestine, cervix, colon, rectum, liver, ovary, pancreas, pituitary adenomas, and secretory adenomas. Blood cancers include lymphomas and leukemia. Exemplary lymphomas include, but are not limited to multiple myeloma, Hodgkin's lymphoma, non-Hodgkin's lymphomas (e.g., cutaneous T cell lymphomas such as Sezary syndrome and Mycosis fungoides, diffuse large cell lymphoma, HTLV-1 associated T cell lymphoma, nodal peripheral T cell lymphoma, extranodal peripheral T cell lymphoma, central nervous system lymphoma, and AIDS-related lymphoma). Exemplary leukemia include, but are not limited to acute and chronic types of both lymphocytic and myelogenous leukemia (e.g. acute lymphocytic or lymphoblastic leukemia, acute myelogenous leukemia, acute myeloid leukemia, chronic myelogenous leukemia, chronic lymphocytic leukemia, T cell prolymphocyte leukemia, adult T cell leukemia, and hairy cell leukemia).

In some embodiments, the cancer is selected from liver cancer, breast cancer, lung cancer, ovarian cancer, esophagus caner, prostate cancer, pancreatic cancer, head and neck cancer, glioblastoma, and multiple myeloma.

In a preferable embodiment, the liver cancer is hepatocellular carcinoma. More preferably, the hepatocellular carcinoma is metastatic, recurrent, refractory or advanced.

In a preferable embodiment, the breast cancer is triple negative breast cancer. More preferably, triple negative breast cancer is metastatic, recurrent, refractory or advanced.

In a preferable embodiment, the breast cancer is estrogen positive breast cancer. More preferably, estrogen positive breast cancer is Her2 negative. More preferably, the estrogen positive breast is metastatic, recurrent, refractory or advanced. Even more preferably, the estrogen positive breast is Her2 negative and metastatic.

In a preferable embodiment, the lung cancer is non-small cell lung carcinoma (NSCLC). More preferably, non-small cell lung carcinoma is metastatic, recurrent, refractory or advanced.

In a preferable embodiment, the lung cancer is small cell lung carcinoma (SCLC). More preferably, small cell lung carcinoma is metastatic, recurrent, refractory or advanced.

In a preferable embodiment, the ovarian cancer is metastatic, recurrent, refractory or advanced.

In a preferable embodiment, the esophagus caner is esophageal squamous cell cancer. More preferable, the esophageal squamous cell cancer is metastatic, recurrent, refractory or advanced.

In a preferable embodiment, the prostate cancer is selected from androgen-dependent, hormone-refractory or castration-resistant prostate cancer. More preferably, the prostate cancer is metastatic castration-resistant prostate cancer.

In a preferable embodiment, the pancreatic cancer is metastatic, recurrent, refractory or advanced.

In a preferable embodiment, the head and neck cancer is metastatic, recurrent, refractory or advanced.

In a preferable embodiment, glioblastoma is glioblastoma multiforme (GBM). More preferably, glioblastoma multiforme is metastatic, recurrent, refractory or advanced.

In a preferable embodiment, multiple myeloma is metastatic, recurrent, refractory or advanced.

### Beneficial Effect

By using the pharmaceutical combination of the compound of formula (I) or a pharmaceutically acceptable salt thereof and one or more additional cancer therapeutic agents, a synergistic effect for treating cancer can be achieved. Moreover, the present inventor has performed a 7-day toxicity study based on cynomolgus monkey model showed that Orin 1001 as the compound of formula (I) or pharmaceutically acceptable salt thereof has a No Observable

Adverse Effect Level (NOAEL) of 150 mg/kg/d, and a 5-fold safety margin amounting to 750 mg/kg/d, which indicates a good safety profile of such compound and thus it can be used in a relatively high dosage without significant side effect like toxicity, either alone or in combination with other cancer therapeutical agent. Accordingly, the pharmaceutical combination according to the invention can be used to effectively treat cancer/tumor by inhibiting tumor growth or killing tumor, for example delaying, arresting, or reversing tumor growth with synergistic effect and good safety.

### EXAMPLES

Some Abbreviations: PO: oral; sc: subcutaneous; iv: intravenous; qod: every other day; qwk: once a week; qd: once a day.

In addition to UPR activation leading to XBP1 splicing by a myriad of cellular insults including genotoxic and microenvironmental stresses, IRE1 can be activated indirectly by a number of small molecules. In the Examples, we explored a number of mechanistically distinct FDA approved or clinical level oncology compounds and surprisingly found that many can induce XPB1 splicing.

The materials and reagents used in the Examples are commercially available. The cells are available from ATCC (American type culture collection). MM.1S (ATCC® CRL-2974™) is human plasmacytoma/myeloma cell; HEK-293 (ATCC® CRL-1573™) is human embryonic kidney cell; H929 (ATCC® CRL-9068™) is human plasmacytoma/myeloma; RPMI 8226 (ATCC® CCL-155™) is human plasmacytoma/myeloma cell; A549 (ATCC® CCL-185™) is human lung epithelial carcinoma cell; HT-29 (ATCC® HTB-38™) is human colorectal adenocarcinoma; MCF7 (ATCC® HTB-22™) is human epithelial mammary gland adenocarcinoma; and Hep G2 (ATCC® HB-8065™) is “Hepatoma”, human hepatocellular carcinoma. Unless stated otherwise, the apparatus and reagents are available from Invitrogen. The compound Orin 1001 may be synthesized according to the process described in WO WO2011/127070.

Examples 1-12 were carried out by the method described below.

Determination of XBP1s level induced by various compounds:

This method applies to any mammalian cell line but was typically applied to human MM1s myeloma cells for EC₅₀ and RPMI 8826 plasmacytoma cells for confirmation of selected compounds. Briefly, cells were grown in standard conditions and spread into 96 well tissue culture plates. Cells were treated with compounds with indicated concentration using serial dilutions. DTT (dithiothreitol) or compounds were added at the same time and cells were harvested after indicated hour's treatment. Cells treated with DTT alone were used as 100% XBP1s positive controls and cell left untreated were used as base line XBP1s level.

Splicing Assay: Total RNA was isolated from cells treated with compounds using Applied Biosystems RNAqueous kit. 1 μg of total RNA was reverse transcribed using Oligo dt (12-18) (Invitrogen). The cDNA was then amplified at 95° C. for 8 min and 30 sec, then 40 cycles at 95° C. for 15 sec and 63° C. for 1 min. Samples were run against a purified standard curve for both spliced and unspliced XBP1, and further normalized to internal house-keeping gene GAPDH.

Probes and primers:

Human XBP1 Forward
(Seq ID No. 1)
GGAAGCCAAGGGGAATGAAGTG
Human XBP1 Reverse
(Seq ID No. 2)
GGAGATGTTCTGGAGGGGTGAC
GAPDH Forward
(Seq ID No. 3)
ATCGTGGAAGGACTCATGACCA
GAPDH Reverse
(Seq ID No. 4)
AGGGATGATGTTCTGGAGAGCC
Human Unspliced XBP1 Probe
(Seq ID No. 5)
5′ CAL FLURO RED-CACGTAG TCTGAGTGCTGCGGACT-BHQ2 3′
Human Spliced XBP1 Probe
(Seq ID No. 6)
5′ FAM-CCTGCACCTGCTGCGGACT-BHQ1 3′
GAPDH Probe
(Seq ID No. 7)
5′ HEX-TCCATGCCATCACTGCCACCCA-BHQ1 3′

### Example 1-12

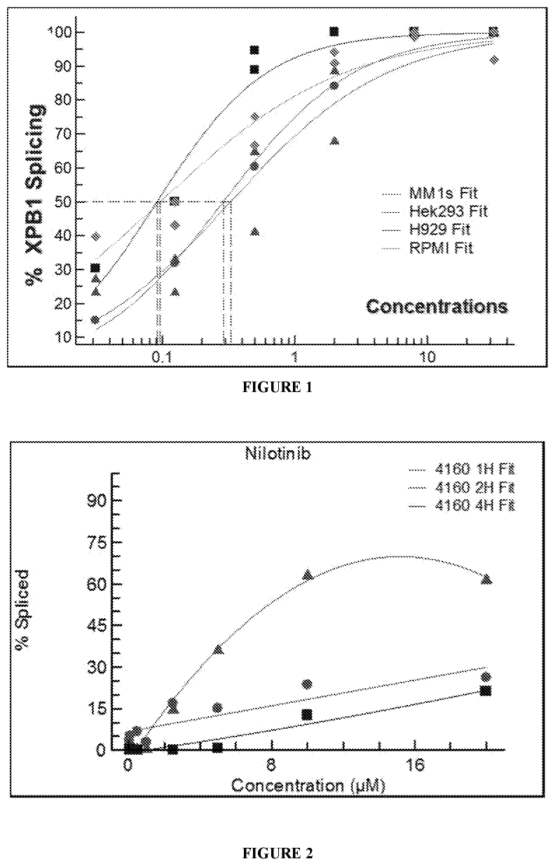

In Examples 1-12, various compounds were tested for their respective induction to cells lines' ER stress measured by XBP1s level and the results are shown in FIGS. 1-12, respectively. We have tested the compounds as several FDA approved kinase inhibitors could induce XBP1s: Nilotinib, Sorafenib, Gefitinib, Dasatinib and the late clinical stage kinase inhibitors Vatalinib and Lestaurtinib; as well as the compounds Temsirolimus (Toricel®), a FDA approved natural product mTOR inhibitor; Vorinostat, a FDA approved HDAC inhibitor; Paclitaxel, a well characterized microtubule disruptor; Gemcitabine, a nucleoside analogue; and 17-AAG, a HSP90 inhibitor and shown that they were all able to induce XBP1s in a time, cell or concentration dependent manner

Example 1 showed that Lestaurtinib enhanced multiple cell lines' ER stress measured by XBP1s level (FIG. 1).

Example 2 showed that Nilotinib enhanced MM1S cell's ER stress measured by XBP1s level (FIG. 2). The same methods were used as above for Lestaurtinib.

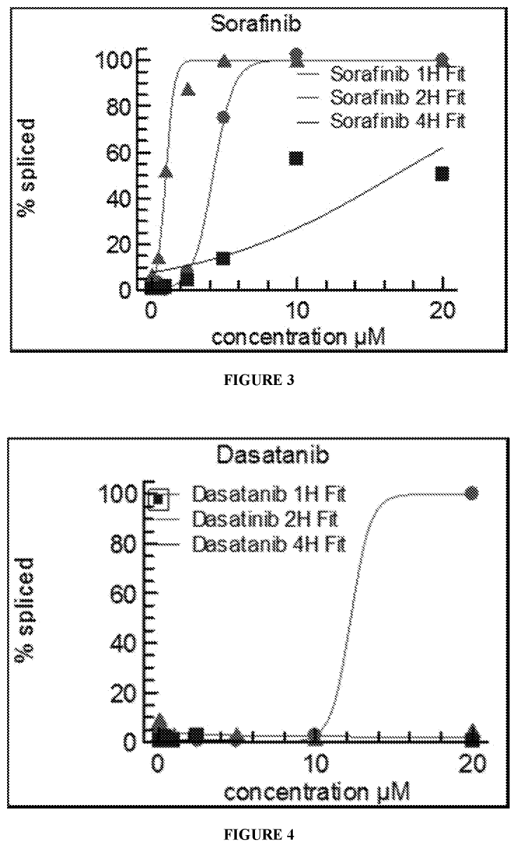

Example 3 showed that Sorafenib enhanced A549 cell lines' ER stress measured by XBP1s level (FIG. 3). The same methods were used as above for Lestaurtinib.

Example 4 showed that Dasatinib enhanced A549 cell lines' ER stress measured by XBP1s level (FIG. 4). The same methods were used as above for Lestaurtinib.

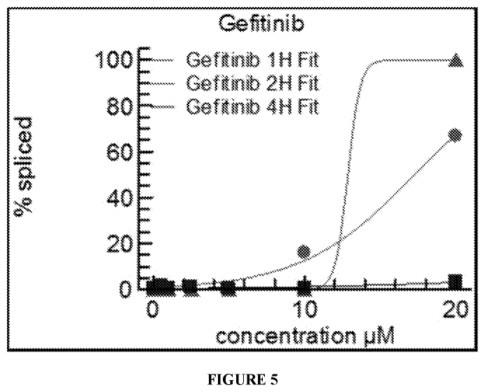

Example 5 showed that Gefitinib enhanced A549 cell lines' ER stress measured by XBP1s level (FIG. 5). The same methods were used as above for Lestaurtinib.

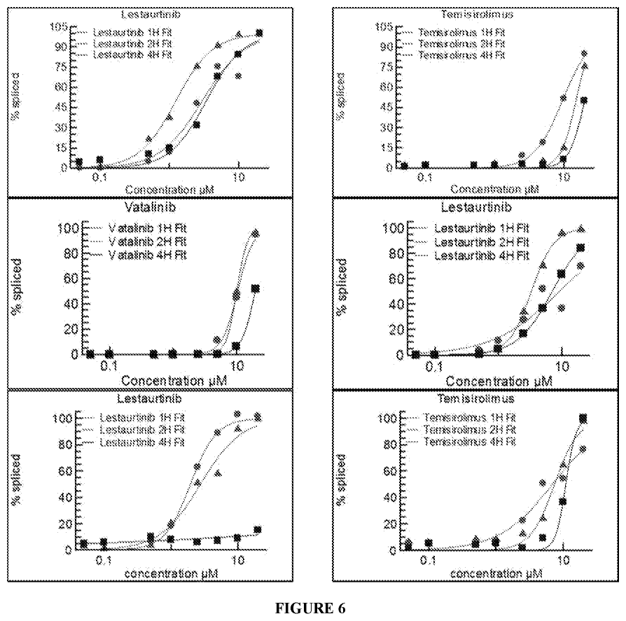

Example 6 showed that Lestaurtinib, temisirolimus, vatalinib enhanced several cell lines' ER stress measured by XBP1s level (FIG. 6). The same methods were used as above for Lestaurtinib.

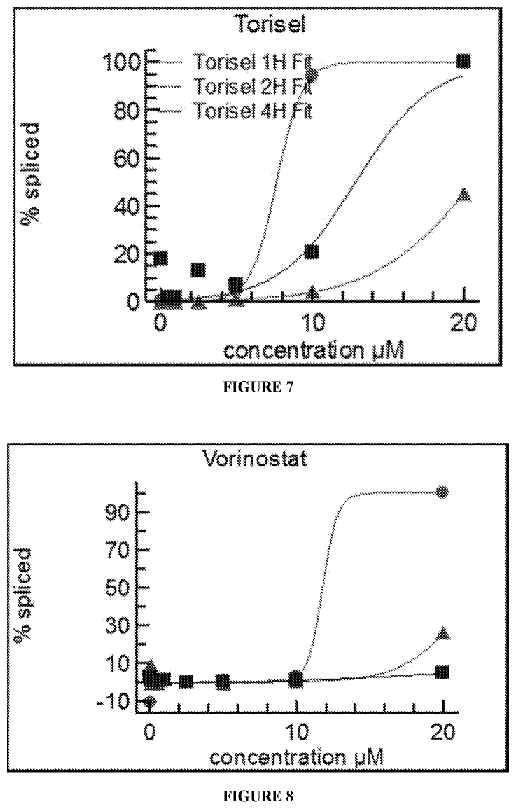

Example 7 showed that Torisel® (Temsirolimus) enhanced A549 cell lines' ER stress measured by XBP1s level (FIG. 7). The same methods were used as above for Lestaurtinib.

Example 8 showed that Vorinostat enhanced HT-29 cell lines' ER stress measured by XBP1s level (FIG. 8). The same methods were used as above for Lestaurtinib.

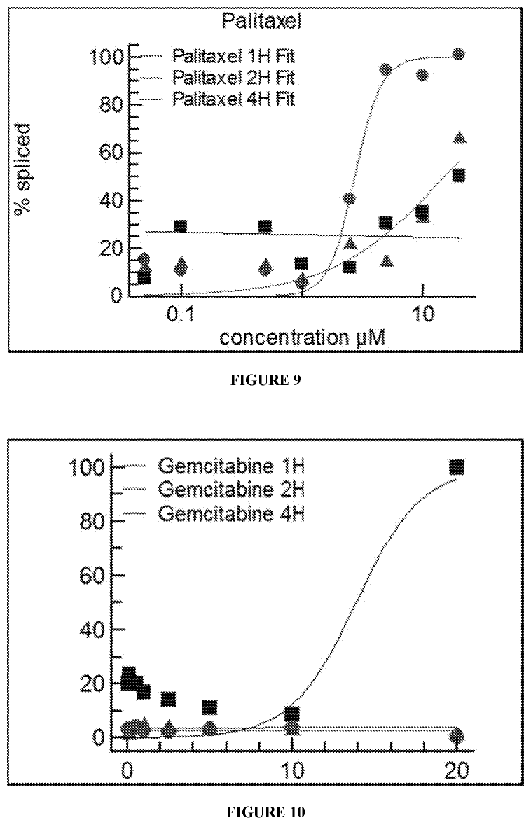

Example 9 showed that Paclitaxel enhanced RPMI 8226 cell lines' ER stress measured by XBP1s level (FIG. 9). The same methods were used as above for Lestaurtinib.

Example 10 showed that Gemcitabine enhanced RPMI 8226 cell lines' ER stress measured by XBP1s level (FIG. 10). The same methods were used as above for Lestaurtinib.

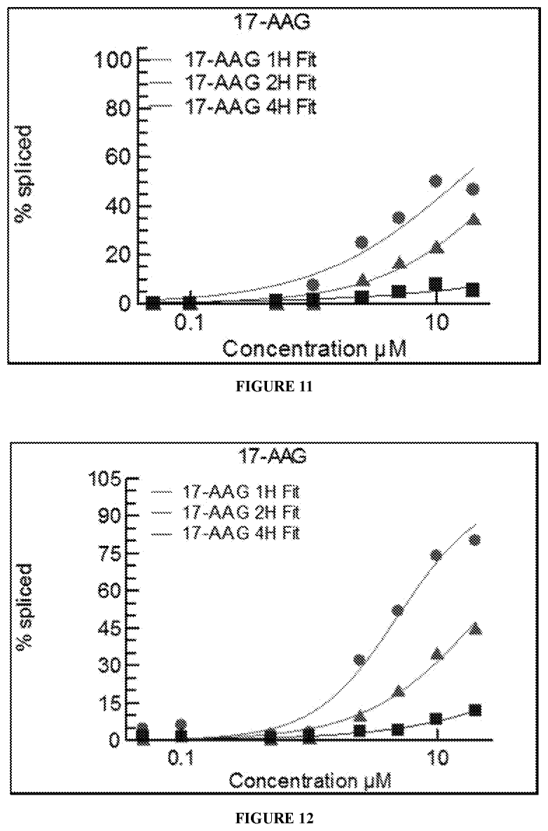

Example 11 showed that 17-AAG enhanced MCF-7 cell lines' ER stress measured by XBP1s level (FIG. 11). The same methods were used as above for Lestaurtinib.

Example 12 showed that 17-AAG enhanced Hepatoma cell lines' ER stress measured by

XBP1s level (FIG. 12). The same methods were used as above for Lestaurtinib.

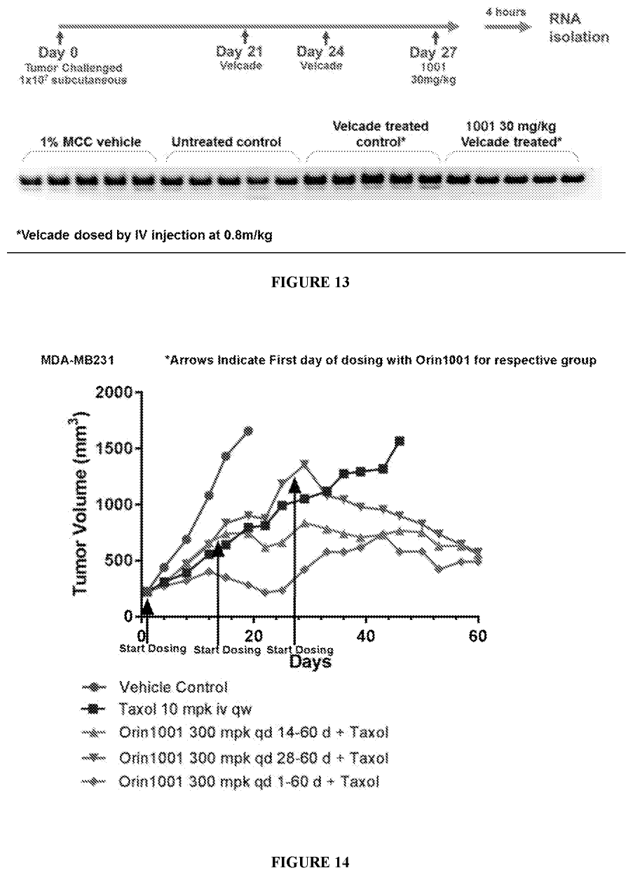

Example 13 showed Intratumoral XBP-1 spliced effect of IRE-1 compound Orin 1001 in Velcade® treated RPMI xenografts (FIG. 13). As shown in FIG. 13, Nude mice with xenografts using RPMI 8226 tumor cells were treated by IV injection of Velcade® at 0.8 mg/kg after the tumor established in 21 days. The mice were treated again on the 24^{th} day with Velcade®. On Day 27, the mice were treated with Orin 1001 at 30 mg/kg PO, four hours later the mice were sacrificed as in Example 25, and tumor tissues were isolated. In a similar way as described for the liver PD test in Example 25, RNA extraction and RT-PCR analysis gave the results as shown FIG. 13. This experiment clearly demonstrated that Velcade® increased the level of XBP1s (lower band in the gel image) in tumor which is an indication of IRE1 activation, and Orin 1001 can inhibit the activity of the activated IRE1. The results suggest a combined strategy of treating cancer patient with Velcade® and Orin 1001 (the compound of formula II herein).

### Examples 14-24

Examples 14-24 were in vivo tests for efficacies of Orin 1001 in combination with other cancer therapeutic agents and the procedures were summarized as follows.

Administration of an IRE-1α inhibitor in combination with a cytotoxic agent or hormone antagonist, VEGF inhibitor, antitumor antibiotic, antimetabolite, platinum coordination complex or alkylating agent may be more effective in inhibiting tumor growth and prevent tumor relapse. A novel, first-in-class IRE-1α inhibitor, Orin 1001, was evaluated in combination with paclitaxel, tamoxifen, Velcade®, sorafenib, eribulin, doxorubicin, 5-FU, carboplatin or cyclophosphamide in mouse tumor xenograft models, which were developed by Charles River Laboratories, CrownBio or WuXi AppTec R&D center, and the studies were performed by Charles River Laboratories, CrownBio or WuXi AppTec R&D center as contracted services. These models include triple negative breast cancer, estrogen positive breast cancer, ovarian carcinoma, pancreatic cancer, head and neck cancer, non-small cell lung cancer, glioblastoma, multiple myeloma and liver cancer. All the therapeutic agents such as paclitaxel, tamoxifen and Velcade® were purchased by Charles River, CrownBio or WuXi AppTec R&D center from commercial sources.

### Example 14: Orin 1001 Inhibits Triple Negative Breast Cancer in Combination with Paclitaxel at Different Tumor Growth Stage

Orin 1001 was administered by oral gavage in combination with paclitaxel in a xenograft mouse model using female NCr nu/nu mice injected subcutaneously with human breast adenocarcinoma MDA-MB231 tumor cells. To evaluate the effect of Orin 1001 in combination with paclitaxel on early, mid or late stage tumor growth, dosing was initiated either on Day 1 (when tumors reached 225-250 mm³), Day 14 or Day 28 of tumor growth. Orin 1001 was administered via oral gavage at 300 mg/kg/day in combination with paclitaxel at 10 mg/kg iv, weekly (n=10/group) for up to 60 days. There were no clinical signs of toxicity with Orin 1001. The results are shown in FIG. 14.

In combination with paclitaxel, Orin 1001 showed significant tumor inhibition compared to paclitaxel alone at all stages of tumor growth. Specifically, Orin 1001 (300 mg/kg) was applied at Day 1, Day 14 and Day 28 respectively in combination with paclitaxel (10 mg/kg) showing the effects of intervene of Orin 1001 at any stage of tumor growth.

As compared to control and paclitaxel alone, the combined use of Orin 1001 and paclitaxel could delay the growth of tumor and the synergistic effect could be seen at every intervening stage like early, middle or late stage, even starting as late as Day 28. Moreover, also compared to control and paclitaxel alone, the growth of tumor could be reversed when Orin 1001 is applied together with paclitaxel at every intervening stage, even starting as late as Day 28. And extended oral dosing of Orin 1001 for up to 60 consecutive days was well tolerated and also resulted in a significant synergistic effect on tumor inhibition.

### Example 15: Orin 1001 Inhibits Triple Negative Breast Cancer in Combination with Paclitaxel Dose Proportionally

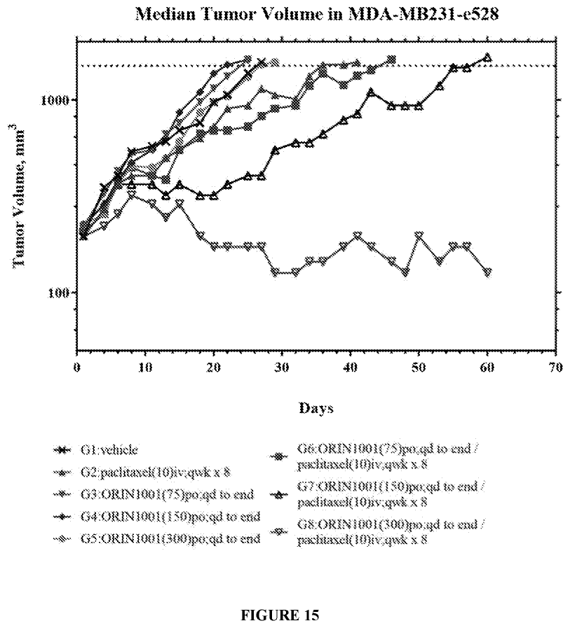

Orin 1001 was administered by oral gavage in combination with paclitaxel in a xenograft mouse model using female NCr nu/nu mice (n=10 in each group) injected subcutaneously with human breast adenocarcinoma MDA-MB231 tumor cells. To evaluate the effect of Orin 1001 in combination with paclitaxel, Orin 1001 was administered via oral gavage at 75, 150 or 300 mg/kg/day in combination with paclitaxel at 10 mg/kg iv, weekly (n=10/group) for up to 60 days. There were no clinical signs of toxicity with Orin 1001. The results are shown in FIG. 15.

In combination with paclitaxel, Orin 1001 showed significant tumor inhibition compared to paclitaxel alone at all stages of tumor growth. Treatment with 300 mg/kg Orin 1001 in combination with paclitaxel resulted in 3 partial regressions and 1 tumor-free survival versus 1 partial regression in the paclitaxel group alone. Particularly, when Orin 1001 was applied at a dose at 150 mg/kg/day or more, the inhibitory effects were much obvious. At every dosing level of combined use of Orin 1001, the growth of tumors could be reversed. Especially, when Orin 1001 was applied at a dose of 300 mg/kg, the tumor growth was almost arrested. And extended oral dosing of Orin 1001 for up to 60 consecutive days was well tolerated and resulted in a significant synergistic effect on tumor inhibition.

### Example 16: Orin 1001 Inhibits Estrogen Positive Breast Cancer in Combination with Tamoxifen

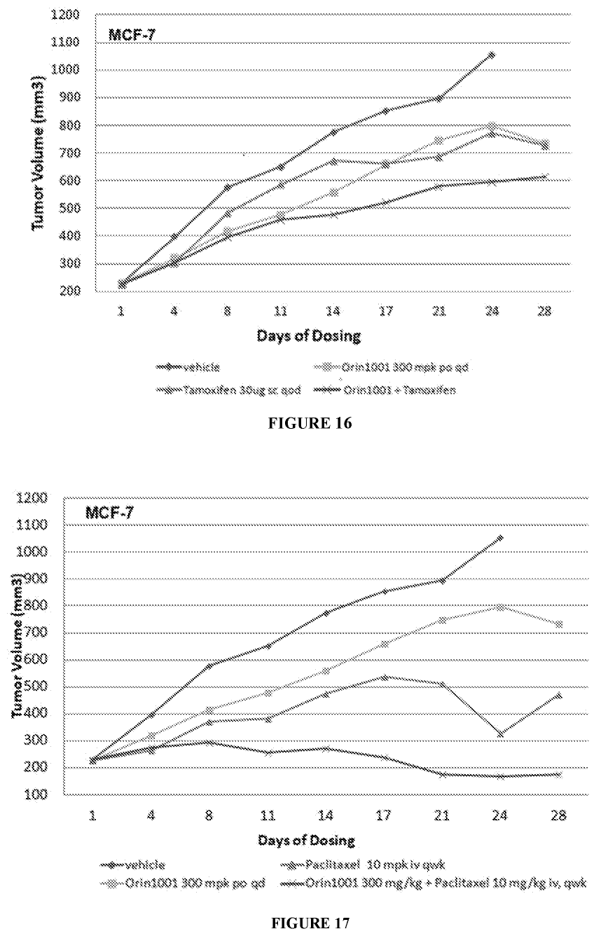

Orin 1001 was administered by oral gavage alone and in combination with tamoxifen using female NCr nu/nu mice injected with human breast adenocarcinoma MCF-7 tumor cells in an orthotopic mouse xenograft model. Three days prior to tumor cell implantation, estrogen pellets were implanted subcutaneously. Tumor cells used for implantation were harvested during log phase growth and implanted into the mammary fat pad. Tumor growth was monitored as the average size approached the target range of 225-250 mm³. Orin 1001 was administered via oral gavage at 300 mg/kg/day and in combination with tamoxifen at 30 μg mg/kg sc, every other day (n=12/group). The results are shown in FIG. 16.

Orin 1001 in combination with tamoxifen showed significant tumor inhibition compared to tamoxifen alone.

### Example 17: Orin 1001 Inhibits Estrogen Positive Breast Cancer in Combination with Paclitaxel

Orin 1001 was administered by oral gavage alone and in combination with paclitaxel using female NCr nu/nu mice injected with human breast adenocarcinoma MCF-7 tumor cells in an orthotopic mouse xenograft model. Three days prior to tumor cell implantation, estrogen pellets were implanted subcutaneously. Tumor cells used for implantation were harvested during log phase growth and implanted into the mammary fat pad. Tumor growth was monitored as the average size approached the target range of 225-250 mm³. Orin 1001 was administered via oral gavage at 300 mg/kg/day and in combination with paclitaxel at 10 mg/kg iv weekly (n=11/group). The results are shown in FIG. 17.

Orin 1001 in combination with paclitaxel showed significant synergistic effects on tumor growth inhibition compared to paclitaxel alone. Specifically, when Orin 1001 was applied in combination with paclitaxel, a synergistic effect could be seen over Orin 1001 or paclitaxel alone and particularly the growth of tumor was almost arrested and then reversed under the combined use.

### Example 18: Orin 1001 Inhibits Ovarian Cancer in Combination with Paclitaxel

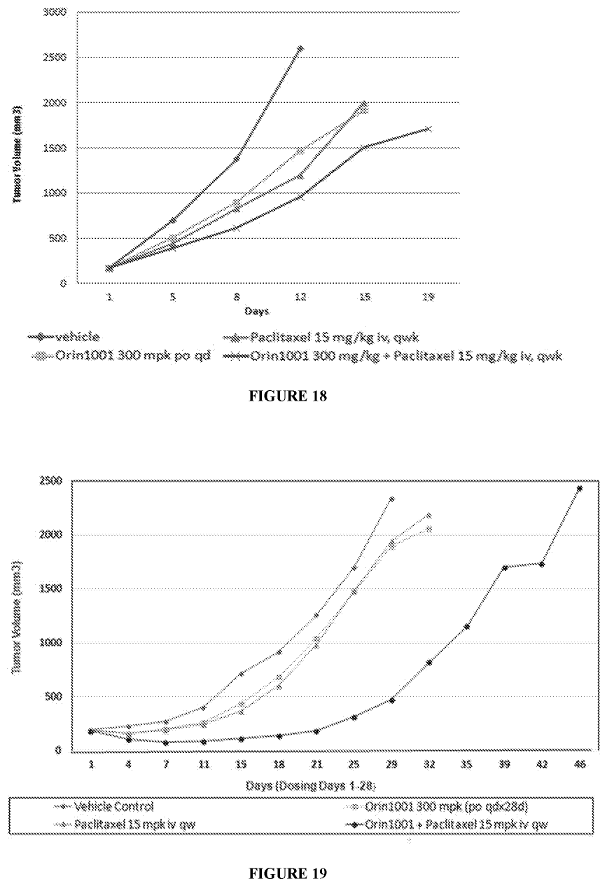

Orin 1001 was administered by oral gavage in a xenograft mouse model using female NCr nu/nu mice injected subcutaneously with human ovarian carcinoma A2780 tumor cells. Animals were assigned to 4 groups (n=6/group); Vehicle control administered by oral gavage for 28 days, Orin 1001 administered via oral gavage at 300 mg/kg/day for 28 consecutive days, paclitaxel administered weekly by iv at 15 mg/kg, or Orin 1001 administered in combination with paclitaxel. The study endpoint was tumor volume of 2000 mm³ or Day 60, whichever came first and the results are shown in FIG. 18.

The percent tumor growth delay was calculated using the following equation: TGD (%)=[T-C/C]×100, where T-C is the difference in time to tumor endpoint from Treated (T) and Control (C). The percent TGD was 29, 40 and 68% for Orin 1001 alone, paclitaxel alone and Orin 1001 in combination with paclitaxel, respectively. According to FIG. 18, it can be seen that in combination with Paclitaxel, 300 mg/kg Orin 1001 showed increased tumor inhibition compared to paclitaxel alone and the reverse of tumor growth was observed under the combined use of Orin 1001 and paclitaxel.

### Example 19: Orin 1001 Inhibits Glioblastoma in Combination with Paclitaxel

Orin 1001 was administered by oral gavage in a xenograft mouse model using female NCr nu/nu mice injected subcutaneously with human glioblastoma U-87 MG tumor cells, a glioblastoma multiforme (GBM) cell line. Animals were assigned to 4 groups (n=6/group); Vehicle control administered by oral gavage for 28 days, Orin 1001 administered via oral gavage at 300 mg/kg/day for 28 consecutive days, paclitaxel administered weekly by iv at 15 mg/kg, or Orin 1001 administered in combination with paclitaxel. The study endpoint was tumor volume of 2000 mm³ or Day 60, whichever came first and the results are shown in FIG. 19.

The percent tumor growth delay was calculated using the following equation: TGD (%)=[T-C/C]×100; where T-C is the difference in time to tumor endpoint from Treated (T) and Control (C). The percent TGD was 13, 17 and 50% for Orin 1001 alone, paclitaxel alone and Orin 1001 in combination with paclitaxel, respectively.

The time to tumor endpoint (TTE) for each animal was further calculated using the following equation: TTE (days)=log 10 (endpoint volume, mm³)-b/m, where b is the intercept and m is the slope of the line obtained by linear regression of the log-transformed tumor growth data set. The TTE was 25.9, 29.3, 30.2 and 38.0 for vehicle control, Orin 1001 alone, paclitaxel alone, Orin 1001 in combination with paclitaxel, respectively.

Tumor growth delay and survival were significantly greater with Orin 1001 in combination with paclitaxel than with paclitaxel alone (p<0.01, Chi Square and Gehan-Breslow-Wilcoxon test). In combination with paclitaxel, 300 mg/kg Orin 1001 showed a marked increase in tumor inhibition compared to paclitaxel alone with 2 animals showing partial regression versus 0 animals in the other treated groups. Specifically, the tumor delay effect was not very significant for Orin 1001 or paclitaxel each alone over the control and the tumor volumes reach the maximum at about Day 30. On the contrary, when they are in combined use, the tumor growth was significantly delayed and the effect of growth reverse was also observed as shown in FIG. 19.

### Example 20: Orin 1001 Inhibits Non-Small Cell Lung Cancer in Combination with Paclitaxel

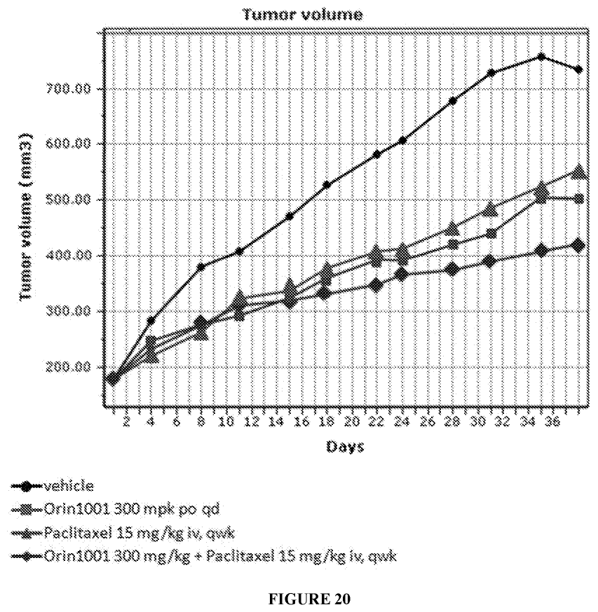

Orin 1001 was administered by oral gavage in a xenograft mouse model using female NCr nu/nu mice injected subcutaneously with A549 human lung carcinoma tumor cells. Animals were assigned to 4 groups (n=10/group); Vehicle control administered by oral gavage for 28 days, Orin 1001 administered via oral gavage at 300 mg/kg/day for 28 consecutive days, paclitaxel administered weekly by iv at 15 mg/kg, or Orin 1001 administered in combination with paclitaxel. The results are shown in FIG. 20.

As shown in FIG. 20, in combination with paclitaxel, when Orin 1001 was used together with paclitaxel, it showed a modest increase in tumor inhibition compared to Orin 1001 or paclitaxel alone.

### Example 21: Orin 1001 Inhibits Liver Cancer in Combination with Sorafenib in Subcutaneous Hep3B Model

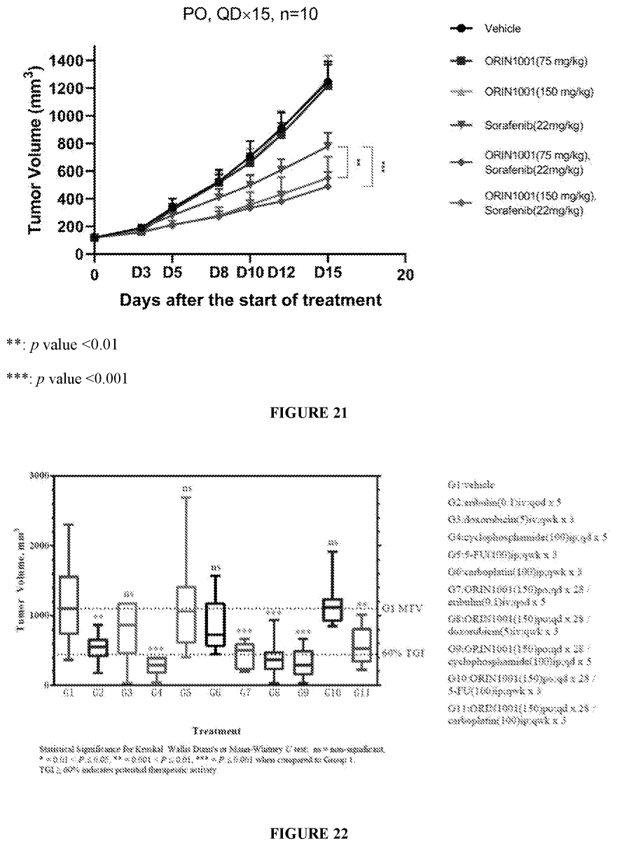

The inhibiting effects of Orin 1001 against liver cancer growth in combination with sorafenib was tested in a subcutaneous Hep3B (ATCC, Manassas, Va., cat #HB-8064) human liver xenograft model with female BALB/c-Nu/Nu mice. Animals received 10×10⁶ Hep3B cells implanted subcutaneously using Matrigel and were assigned to 6 groups (n=10/group). Sorafenib was purchased from Bide Pharmatech LTD and formulated as a solution Cremophor® EL/ethanol (50:50). Orin 1001 was formulated as suspension with cellulose microcrystalline and sucrose in purified water. Sorafenib was given via oral gavage at a dose of 22 mg/kg for 15 days, Orin 1001 via oral gavage at 75 mg/kg or 150 mg/kg for 15 days, or Orin 1001 administered in combination with sorafenib. Tumor volumes were measured using calipers. According to FIG. 21, a significant statistical result was shown from the 8^{th} until 15^{th} day for both of the combination dosing groups, compared with either single treatment group. When compared with sorafenib single treatment group, the p value for Orin 1001 (150 mg/kg) combination group is 0.004 (<0.01), while the p value for Orin 1001 (75 mg/kg) combination group is <0.0001.

### Example 22: Orin 1001 Inhibits Liver Cancer in Combination with Sorafenib in Orthotopic Hep3B-Luc Model

The in vivo anti-tumor efficacy of Orin 1001 in combination with Sorafenib in orthotopic Hep3B-luc human liver xenograft model in female BALB/c nude mice was tested. The tumor was inoculated by injecting Hep3B-Luc cells (established by WuXi AppTec R&D center) mixed with BD Matrigel in 20 μl (PBS:Matrigel=1:1) into the left lobe of the liver. Orin 1001 was formulated at 16 mg/ml with Orin 1001 dissolved with 1% (w/v) cellulose microcrystalline in 50% (w/v) sucrose in purified water. Sorafenib was formulated with Cremophor® EL/ethanol (1:1) and purified water to form a solution of 9 mg/ml. Animals were assigned to 4 groups (n=10/group): Vehicle control administered by oral gavage for 28 days, Orin 1001 or sorafenib administered via oral gavage for 28 consecutive days at a dose of 80 mg/kg/day or 45 mg/kg/day respectively, or Orin 1001 administered in combination with sorafenib. Tumor weight was measured at the termination of study. T/C_{weight} value (in percent) was calculated using the formula: T/C_{weight}%=T_{weight}/C_{weight}×100%, while T_{weight} and C_{weight} were the mean tumor weights of the treated and the vehicle control groups, respectively. The potential synergistic effect between Orin 1001 and Sorafenib was analyzed by two-way ANOVA. For the combination article to be considered to have synergistic effect, T/C_{weight}% of the combination group must be less than two single drug groups and p value (two-way ANOVA) less than 0.05. The result shown in Table 1 indicated a synergistic effect between Orin 1001 and sorafenib.

TABLE 1
Synergistic effect of Orin1001 and sorafenib in orthotopic Hep3B-luc
liver xenograft model

| | | | p value | p value |
|---|---|---|---|---|
| | Tumor Weight | T/C_{weight} | (one-way | (two-way |
| Treatment | (g) at day 28 | (%) | ANOVA) | ANOVA) |
| Vehicle | 4.76 ± 2.06 | — | — | |
| ORIN1001 (80 mg/kg) | 2.68 ± 1.69 | 56 | 0.100 | |
| Sorafenib (45 mg/kg) | 0.91 ± 0.29 | 19 | 0.001 | |
| ORIN1001 + Sorafenib | 0.73 ± 0.41 | 15 | 0.001 | 0.037 |
| (80 mg/kg + 45 mg/kg) | | | | |

### Example 23: Orin 1001 Inhibits Liver Cancer in Combination with Sorafenib in Subcutaneous HUH-7 Model

Female BALB/c nude mice were subcutaneously inoculated with human liver HUH-7 cells (from JCRB) to establish a xenograft model of liver cancer. The scheduled administration cycle was 28 days. Animals were assigned into 4 groups (n=10/group): one vehicle group, two single treatment groups (Orin 1001 administered at 80 mg/kg/day q.d. for 28 days; Sorafenib administered following a dose regimen of 18.25 mg/kg/day q.d. for 5 days, then 25 mg/kg/day q.d. for 3 days, and then 45 mg/kg/day q.d. for 20 days, consecutively) and one combination treatment group (sorafenib administered following a dose regimen of 18.25 mg/kg/day q.d. for 5 days, then 25 mg/kg/day q.d. for 3 days, and then 45 mg/kg/day q.d. for 20 days, consecutively, whilst combined with Orin 1001 administered q.d. at a dose of 80 mg/kg/day for 28 days). All vehicle or administration treatments were given via oral gavage. Sorafenib was formulated with Kolliphor® EL/ethanol (2/1, v/v) and water. Orin 1001 was dissolved with cellulose microcrystalline and sucrose in purified water to form as suspension. The mice were euthanized if a tumor volume achieved over 2000 mm³. Otherwise the experiment ended on the 6^{th} day after the last administration. A result of animal survival time was shown in Table 2. Orin 1001 (80 mg/kg) combined with sorafenib (18.25/25/45 mg/kg) compared with the control group significantly prolonged survival (ILS (increase of life span)=35%, p=0.035).

TABLE 2
Synergistic effect of animal survival time of Orin1001 and sorafenib
in HUH-7 model

| | Median Survival | 95% | | |
|---|---|---|---|---|
| | Time (MST)^{a}, | Confidence | | |
| Treatment | day | Interval^{a} | ILS (%)^{b} | P Value |
| Group 1: | 20 | 17.0-23.0 | — | — |
| Vehicle Control | | | | |
| Group 2: | 20 | 17.0-23.0 | 0 | 0.492 |
| Orin 1001 80 mg/kg | | | | |
| Group 3: | 24 | 20.2-27.8 | 20 | 0.689 |
| Sorafenib | | | | |
| 18.25/25/45 mg/kg | | | | |
| Group 4: | | | | |
| Orin1001 80 mg/kg + | 27 | 21.8-32.2 | 35 | 0.035 |
| Sorafenib | | | | |
| 18.25/25/45 mg/kg | | | | |

^{a}The median survival time (MST) and corresponding 95% confidence interval were calculated by Kaplan-Meier method and Log rank test was performed. P < 0.05 was considered to be a significant difference.
^{b}ILS % = (1 − C_{MST}/T_{MST}) × 100%; C_{MST}: median survival time of vehicle group (group 1), T_{MST}: median survival time of each administration group, i.e. group 2-4.

### Example 24: Orin 1001 Inhibits Triple Negative Breast Cancer in Combination with Eribulin, Doxorubicin, Cyclophosphamide, 5-FU or Carboplatin

In Example 24, Orin 1001 was used in combination with eribulin, doxorubicin, cyclophosphamide, 5-FU or carboplatin to treat triple negative breast cancer. The tests were performed on MDA-MB231-e551 xenograft model and the protocol was listed in Table 3.

TABLE 3
Protocol Design

| | | Treatment Regimen 1 | | | | Treatment Regimen 2 | | | |
|---|---|---|---|---|---|---|---|---|---|
| Group | n | Agent | mg/kg | Route | Schedule | Agent | mg/kg | Route | Schedule |
| 1 | 11 | vehicle | — | po | qd × 28 | — | — | — | — |
| 2 | 11 | eribulin | 0.1 | iv | qod × 5 | — | — | — | — |
| 3 | 11 | doxorubicin | 5 | iv | qwk × 3 | — | — | — | — |
| 4 | 11 | cyclophosphamide | 100 | ip | qd × 5 | — | — | — | — |
| 5 | 11 | 5-FU | 100 | ip | qwk × 3 | — | — | — | — |
| 6 | 11 | carboplatin | 100 | ip | qwk × 3 | — | — | — | — |
| 7 | 11 | ORIN1001 | 150 | po | qd × 28 | eribulin | 0.1 | iv | qod × 5 |
| 8 | 11 | ORIN1001 | 150 | po | qd × 28 | doxorubicin | 5 | iv | qwk × 3 |
| 9 | 11 | ORIN1001 | 150 | po | qd × 28 | cyclophosphamide | 100 | ip | qd × 5 |
| 10 | 11 | ORIN1001 | 150 | po | qd × 28 | 5-FU | 100 | ip | qwk × 3 |
| 11 | 11 | ORIN1001 | 150 | po | qd × 28 | carboplatin | 100 | ip | qwk × 3 |

In the tests of Example 24, 5-FU (5-fluorouracil) was diluted with sterile saline (0.9% NaCl) to a concentration of 10 mg/mL; carboplatin was diluted to 10 mg/mL with 5% dextrose in water; Orin 1001 was formulated in 1% microcrystalline cellulose in a sucrose aqueous solution as a suspension of 15 mg/mL; cyclophosphamide was diluted with sterile saline a concentration of 10 mg/mL; eribulin was diluted with sterile saline (0.9% NaCl) to a concentration of 0.01 mg/mL; doxorubicin was diluted with sterile saline (0.9% NaCl) to a concentration of 0.5 mg/mL

The efficacy of Orin 1001 in combination with eribulin, doxorubicin, cyclophosphamide, 5-FU or carboplatin was tested in the MDA-MB231-e551 human triple negative breast cancer xenograft model using female athymic nude mice (Crl:NU(Ncr)-Foxnlnu, Charles River). Tumor xenografts were initiated with MDA-MB-231 human breast carcinoma cells cultured in RPMI-1640 medium containing 10% fetal bovine serum, 100 units/mL penicillin G, 100 g/mL streptomycin sulfate, 2 mM glutamine and 25 μg/mL gentamicin. Cells were cultured in tissue culture flasks in a humidified incubator at 37° C., in an atmosphere of 5% CO₂ and 95% air. The tumor cells used for implantation were harvested during log phase growth and resuspended in phosphate buffered saline (PBS) at a concentration of 5×10⁷ cells/mL. On the day of implantation, each test mouse received 5×10⁶ MDA-MB231 cells (0.1 mL cell suspension) implanted subcutaneously in the right flank and tumor growth was monitored as the average size approached the target range of 225-275 mm³. Twenty-nine days later, designated as Day 1 of the study, the animals were sorted into eleven groups (n=11/group). Mice were dosed according to the protocol shown in Table 3. Group 1 was vehicle, groups 2-6 were single dosed, and groups 7-11 were combined dosed: Orin 1001 administered with eribulin, doxorubicin, cyclophosphamide, 5-FU or carboplatin, respectively. All vehicle and Orin 1001 doses were administered via oral gavage (p.o.) daily for twenty-eight days (qd×28). Eribulin was administered at 0.1 mg/kg intravenously (i.v.) every other day for a total of five doses (qod×5). Doxorubicin was administered at 5 mg/kg i.v. once weekly for three weeks (qwk×3). Cyclophosphamide was administered at 100 mg/kg intraperitoneally (i.p.) once daily for five days (qd×5). 5-FU was administered at 100 mg/kg i.p. qwk×3. Carboplatin was administered at 100 mg/kg i.p. qwk×3. Tumors were measured using calipers twice per week, and each animal was euthanized when its tumor reached the endpoint volume of 2000 mm³ or at the end of the study (Day 30), whichever came first.

MTV(n) was defined as the median tumor volume on the last day of the study in the number of animals remaining (n) whose tumors had not attained the endpoint volume. Tumor growth inhibition (TGI) analysis was used to evaluate the difference in median tumor volumes (MTVs) of treated and control animals. For this study, the endpoint for determining TGI was Day 20, which was the last day that all evaluable control mice remained in the study. The MTV (n), the median tumor volume for the number of animals in groups, n, on the day of TGI analysis, was determined for each group. Percent tumor growth inhibition (% TGI) was defined as the difference between the MTV of the designated control group and the MTV of the drug-treated group, expressed as a percentage of the MTV of the control group:

$$\% TGI = ( \frac{{MTV}_{control} - {MTV}_{drug - treated}}{{MTV}_{control}} ) \times 100 = [ 1 - ( {MTV}_{drug - treated} / {MTV}_{control} ) ] \times 100$$

The data set for TGI analysis included all animals in a group, except those that died due to treatment-related (TR) or non-treatment-related (NTR) causes prior to the day of TGI analysis. A TGI of at least 60% in this assay was considered to be potentially therapeutically active. Statistical analyses of the differences between Day 20 median tumor volumes (MTVs) of control and treated groups were accomplished using the Mann-Whitney U-test. For statistical analyses, two-tailed tests were conducted at significance level P=0.05. Prism summarized test results as not significant (ns) at P>0.05, significant (symbolized by “*”) at 0.01<P<=0.05, very significant (“**”) at 0.001<P<=0.01, and extremely significant (“***”) at P<=0.001.

According to FIG. 22, group 7 (Orin 1001/eribulin), group 8 (Orin 1001/doxorubicin) and group 11 (Orin 1001/carboplatin) exhibited additive or synergistic effects compared with corresponding single dosed groups, showing that the addition of Orin 1001 significantly enhanced each agent's antitumor effect. For example, group 8 (Orin 1001/doxorubicin) and group 11 (Orin 1001/carboplatin) showed significant improvement respectively, when compared with corresponding single treatment group (group 3, group 6), for either TGI or MTV result. Group 7 (Orin 1001/eribulin) exhibited an additive effect for TGI result and a synergistic effect for MTV result, when compared with group 2.

### Example 25: Orin 1001 was Used in Combination with Other Therapeutic Agents

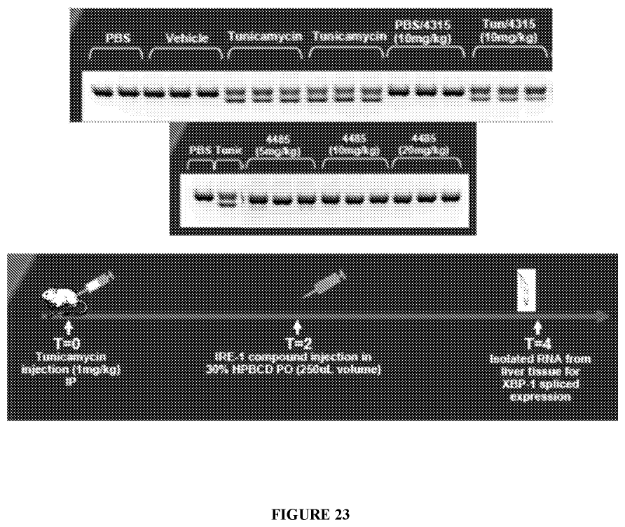

Example 25 was related to comparative PD/PK data of Orin 1001 VS compound 4315, as its structure shown below, which is an earlier lead of this series of compounds. Tunicamycin was used herein to activate IRE1, then Orin 1001 (also called 4485) or 4315 were given to inhibit the activating effect. Orin 1001 demonstrated potent in vivo potency inhibiting IRE1α in our liver PD screening assay. The results are shown in FIG. 23. Each gel panel represents one mouse liver sample as in the figure.

BALB/c mice were injected intraperitoneally with 100 microliters at an equivalent dose of 1 mg/kg tunicamycin solution. Two hours after tunicamycin injection, mice were dosed with compound of interest either PO or IV. Following 2 hours for PO delivery of compound, mice were euthanized according to IACUC protocols using CO₂ from a compressed air source. A 1 cm³ fragment of the liver for homogenization and extraction of the RNA were collected for further analysis. Total RNA is harvested from cells or tissue using TRI_{zol} according to the manufacturer's procedures. After ethanol precipitation and resuspension of the RNA, RiboGreen (Invitrogen) is used to quantify the yield and normalize the RNA concentration in the source tube containing isolated RNA. RT-PCR is performed by Oligo(dT) priming, and SuperScript II (Invitrogen) transcription using the Amplitaq Gold Kit (Applied Biosystems) according to the manufacturer's protocols. Primers for human XBP-1 are 5_-CCTGGTTGCTGAAGAGGAGG-3_ (forward, Seq ID No. 8) and 5_-CCATGGGGAGATGTTCTGGAG-3_ (reverse, Seq ID No. 9), and for mouse are 5_-ACACGCTTGGGAATGGACAC-3_ (forward, Seq ID No. 10) and 5_-CCATGGGAAGATGTTCTGGG-3 (Seq ID No. 11). All DNA oligos were purchased from IDT DNA Technologies. PCR is run on a Bio-Rad PTC-100 96-well thermocycler with heating at 94° C. for 30 s, annealing at 58° C. for 30 s, and polymerizing at 72° C. for 30 s for 35 cycles. Reactions are run on 4% precast NuSieve gels from Cambrex and visualized by ethidium bromide staining and UV excitation.

Accuprime kit (12339-024, Invitrogen)

mXBP-1 458
(Seq ID No. 12)
5′-GAGGCCAAGGGGAGTGGA-3′
(custom order, IDT)
mXBP-1 572
(Seq ID No. 13)
5′-AGATGTTCTGGGGAGGTGACAACT-3′
(custom order, IDT)

mGAPDH 548 (custom order, IDT)

mXBP-1 524 UnSp
(Seq ID No. 14)
5′Tex-CACATAGTCTGAGTGTGCTG-3′BHQ-2
(custom order, Biosearch Technologies)
mXBP-1 580 Sp
(Seq ID No. 15)
5′FAM-CCTGCACCTGCTGCGGACT-3′BHQ-1
(custom order, Biosearch Technologies)

mGAPDH 608 5′HEX/3′BHQ-1 (custom order, Biosearch Technologies)

yeast tRNA (54016, Invitrogen)

thin-wall 96 well RTq PCR plate.

All PK experiments are standard tests that performed in either WuXi PharmaTech or Charels River Laboratory. 4315 and 4485 (Orin 1001) were dosed PO as a suspension in 1% microcellulose (Sigma) and 50% Sucrouse (Sigma).

As shown in FIG. 23, as an example, Orin 1001 has an ED₅₀ less than 2 mg/kg PO vs 4315 which has an ED₅₀>10 mg/kg. 4315 ED₅₀ was determined to be 50 mg/kg in a separate experiment. Compound 4315 is disclosed in WO2011/127070 A2 as compound B, a preferred IRE1α inhibitor. In the figure, top panel when labeled as PBS/4315 or Tun/4315, they meant the mice was either dosed with PBS buffer or tunicamycin to active the IRE1α so that XBP1s was observed as illustrated in the lower panel, and then 4315 was dosed to test its inhibitory effect on IRE1α. In the middle panel, all the 4485 dosing group mice were dosed firstly with tunicamycin.

Orin 1001 (4485) also has much improved oral bioavilability as shown in the Table 4 cross all tested spices.

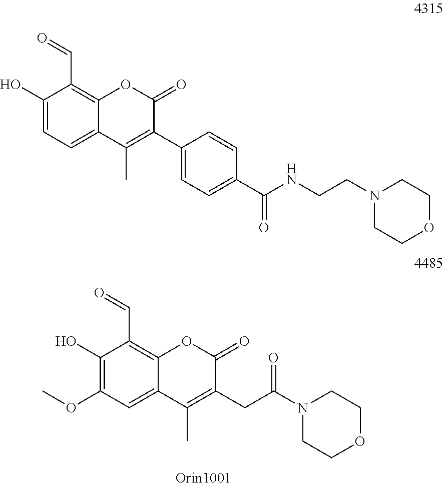 [SMILES: Cc1c(-c2ccc(C(=O)NCCN3CCOCC3)cc2)c(=O)oc2c(C=O)c(O)ccc12 ; COc1cc2c(C)c(CC(=O)N3CCOCC3)c(=O)oc2c(C=O)c1O]

TABLE 4
Oral bioavailability test

| | 4315 oral bioavailability | Orin1001 oral bioavilability |
|---|---|---|
| Mouse | 23% (5 mg/kg dose) | 63% (10 mg/kg dose) |
| Rat | 36% (10 mg/kg dose) | 52% (10 mg/kg dose) |
| Dog | 17% (10 mg/kg dose) | 68.5% (4 mg/kg dose) |

1. A pharmaceutical combination, comprising
(a) a compound of formula (I) or a pharmaceutically acceptable salt thereof, and (b) one or more additional cancer therapeutic agents: 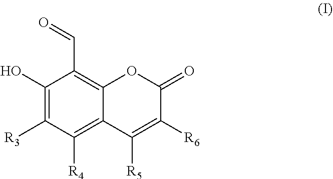
wherein
R₃ and R₄ are independently hydrogen or C₁₋₆ alkoxyl, which is optionally substituted with one or more substituents selected from the group consisting of (1) C₁-C₆ hydrocarbon chain containing N or O atom, and (2) C₃₋₁₀ cycloalkyl, which optionally contains 1 or 2 heteroatoms independently selected from the group consisting of N, O, and S;
R₅ is hydrogen, C₁₋₆ alkyl, C₁₋₆ alkoxyl, or C₁₋₆ alkylamino;
R₆ is C₁₋₆ alkyl, which is substituted with 1, 2 or 3 substituents independently selected from the group consisting of C₁₋₆ alkoxyl, C₁₋₆ hydroxylalkyl, C₁₋₆ alkoxyl C₁₋₆alkyl, 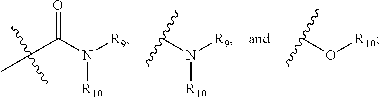
R₉ and R₁₀ are independently hydrogen; C₁₋₆ alkyl; C₁₋₆ alkoxyl C₁₋₆ alkyl; perfluoro C₁₋₆alkoxyl C₁₋₆alkyl; or
R₉ and R₁₀ together with the nitrogen atom to which they are attached form a heterocycle containing 1, 2, 3, or 4 heteroatoms independently selected from the group consisting of N, O, and S, and the heterocycle is optionally substituted with 1, 2, or 3 substituents independently selected from the group consisting of C₁₋₆ alkyl, C₁₋₆ alkylamino, C₁₋₆ alkoxyl.

2. The pharmaceutical combination according to claim 1, wherein
the additional cancer therapeutic agent has at least one of the following features:
(1) inducing ER stress;
(2) inducing or up-regulating IRE-1α expression;
(3) inducing or up-regulating XBP1 splicing; and
(4) being less effective when IRE-1α is expressed.

3. The pharmaceutical combination according to claim 1, wherein the compound of formula (I) or the pharmaceutically acceptable salt thereof and one or more additional cancer therapeutic agents are administered simultaneously, separately or sequentially.

4. The pharmaceutical combination according to claim 1, wherein
the compound of formula (I) has formula (II)

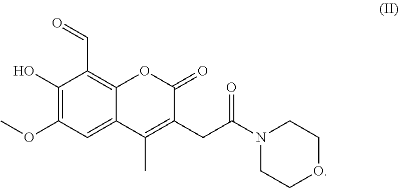 [SMILES: COc1cc2c(C)c(CC(=O)N3CCOCC3)c(=O)oc2c(C=O)c1O]

5. The pharmaceutical combination according to claim 1, wherein
the pharmaceutical combination is in the form of a pharmaceutical composition or a kit.

6. The pharmaceutical combination according to claim 1, wherein
the additional cancer therapeutic agent is selected from the group consisting of
cytotoxic chemotherapeutic agents; antimetabolites; antimitotic agents; alkylating agents; DNA damaging agents; antitumor antibiotics; platinum coordination complexes; proteasome inhibitors; HSP90 inhibitors; hormones and hormone analogs; aromatase inhibitors; fibrinolytic agents; antimigratory agents; antisecretory agents; immunosuppressives; anti-angiogenic compounds and vascular endothelial growth factor inhibitors; fibroblast growth factor inhibitors; epidermal growth factor receptor inhibitors; antibodies; checkpoint inhibitors; cell cycle inhibitors and differentiation inducers; mTOR inhibitors; corticosteroids; growth factor signal transduction kinase inhibitors; mitochondrial dysfunction inducers; caspase activators; chromatin disruptors and DNA repair enzyme inhibitors; HDAC inhibitors; Bcr-Abl inhibitors; FMS-like tyrosine kinase 3 (Flt3) inhibitors; and preferably selected from the group consisting of lestaurtinib, nilotinib, sorafenib, dasatinib, gefitinib, temisirolimus, vatalinib, Torisel®, vorinostat, paclitaxel, gemcitabine, 17-AAG, Velcade®, tamoxifen, temozolomide; or selected from the group consisting of sorafenib, eribulin, cyclophosphamide, 5-fluorouracil, carboplatin, doxorubicin, anastrozole; more preferably selected from the group consisting of paclitaxel, Velcade®, tamoxifen and temozolomide; or selected from the group consisting of sorafenib, eribulin, cyclophosphamide, 5-fluorouracil, carboplatin, doxorubicin.

7. The pharmaceutical combination according to claim 6, wherein
the additional cancer therapeutic agent is sorafenib;
or
the additional cancer therapeutic agent is selected from the group consisting of
(i) microtubule disruptor, wherein said microtubule disruptor is selected from taxane and eribulin, and said taxane is selected from paclitaxel, docetaxel, cabazitaxel and albumin-bound paclitaxel, preferably paclitaxel and docetaxel, more preferably paclitaxel;
(ii) cyclophosphamide;
(iii) 5-fluorouracil;
(iv) carboplatin;
(v) doxorubicin;
or
the additional cancer therapeutic agent is selected from the group consisting of
(i) microtubule disruptor, wherein said taxane is selected from paclitaxel, docetaxel, cabazitaxel and albumin-bound paclitaxel, preferably paclitaxel and docetaxel, more preferably paclitaxel;
(ii) aromatase inhibitor, wherein said aromatase inhibitor is selected from letrozole and anastrozole;
(iii) tamoxifen;
or
the additional cancer therapeutic agent is taxane, wherein said taxane is selected from paclitaxel, docetaxel, cabazitaxel and albumin-bound paclitaxel, preferably paclitaxel and docetaxel, more preferably paclitaxel.

8.-10. (canceled)

11. A kit or a pharmaceutical composition, comprising the pharmaceutical combination according to claim 1.

12. A method for treating cancerous tumor, comprising administering a subject in need thereof an effective amount of the kit or pharmaceutical composition according to claim 11.

13. The method according to claim 12, wherein the cancerous tumor is selected from the group consisting of liver cancer, triple negative breast cancer, estrogen positive breast cancer, ovarian carcinoma, pancreatic cancer, head and neck cancer, non-small cell lung cancer, glioblastoma, for example glioblastoma multiforme, and multiple myeloma.

14. The method according to claim 12, wherein the compound of formula (I) or the pharmaceutically acceptable salt thereof and one or more additional cancer therapeutic agents are administered simultaneously, separately or sequentially.

15. The method according to claim 12, wherein
the cancerous tumor is liver tumor, preferably hepatocellular carcinoma; and
the additional cancer therapeutic agent is sorafenib.

16. The method according to claim 12, wherein
the cancerous tumor is breast cancer, preferably triple negative breast cancer; and
the additional cancer therapeutic agent is selected from
(i) microtubule disruptor, wherein said microtubule disruptor is selected from taxane and eribulin, and said taxane is selected from paclitaxel, docetaxel, cabazitaxel and albumin-bound paclitaxel, preferably paclitaxel and docetaxel, more preferably paclitaxel;
(ii) cyclophosphamide;
(iii) 5-fluorouracil;
(iv) carboplatin;
(v) doxorubicin;
or
the cancerous tumor is breast cancer, preferably estrogen positive breast cancer, more preferably Her2 negative and estrogen positive metastatic breast cancer; and
the additional cancer therapeutic agent is selected from
(i) microtubule disruptor, wherein said taxane is selected from paclitaxel, docetaxel, cabazitaxel and albumin-bound paclitaxel, preferably paclitaxel and docetaxel, more preferably paclitaxel;
(ii) aromatase inhibitor, wherein said aromatase inhibitor is selected from letrozole and anastrozole;
(iii) tamoxifen;
or
the cancerous tumor is esophagus cancer (preferably esophageal squamous cell cancer), ovarian cancer, non-small cell lung cancer, or glioblastoma; and
the additional cancer therapeutic agent is taxane, wherein said taxane is selected from paclitaxel, docetaxel, cabazitaxel and albumin-bound paclitaxel, preferably paclitaxel and docetaxel, more preferably paclitaxel.

17.-18. (canceled)

19. A method for enhancing the efficacy of a cancer therapeutic agent,
comprising applying the compound of formula (I) or a pharmaceutically acceptable salt thereof in combination with the cancer therapeutic agent, 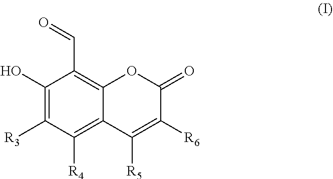
wherein
R₃ and R₄ are independently hydrogen or C₁₋₆ alkoxyl, which is optionally substituted with one or more substituents selected from the group consisting of (1) C₁-C₆ hydrocarbon chain containing N or O atom, and (2) C₃₋₁₀ cycloalkyl, which optionally contains 1 or 2 heteroatoms independently selected from the group consisting of N, O, and S;
R₅ is hydrogen, C₁₋₆ alkyl, C₁₋₆ alkoxyl, or C₁₋₆ alkylamino;
R₆ is C₁₋₆ alkyl, which is substituted with 1, 2 or 3 substituents independently selected from the group consisting of C₁₋₆ alkoxyl, C₁₋₆ hydroxylalkyl, C₁₋₆ alkoxyl C₁₋₆alkyl, 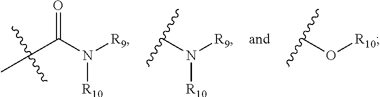
R₉ and R₁₀ are independently hydrogen; C₁₋₆ alkyl; C₁₋₆ alkoxyl C₁₋₆ alkyl; perfluoro C₁₋₆ alkoxyl C₁₋₆alkyl; or
R₉ and R₁₀ together with the nitrogen atom to which they are attached form a heterocycle containing 1, 2, 3, or 4 heteroatoms independently selected from the group consisting of N, O, and S, and the heterocycle is optionally substituted with 1, 2, or 3 substituents independently selected from the group consisting of C₁₋₆ alkyl, C₁₋₆ alkylamino, C₁₋₆ alkoxyl.

20. The method according to claim 19, wherein the cancer therapeutic agent has at least one of the following features:
(1) inducing ER stress;
(2) inducing or up-regulating IRE-1α expression;
(3) inducing or up-regulating XBP1 splicing; and
(4) being less effective when IRE-1α is expressed.

21. The method according to claim 19, wherein the compound of formula (I) or the pharmaceutically acceptable salt thereof and one or more additional cancer therapeutic agents are administered simultaneously, separately or sequentially.

22. The method according to claim 19, wherein
the compound of formula (I) has formula (II)

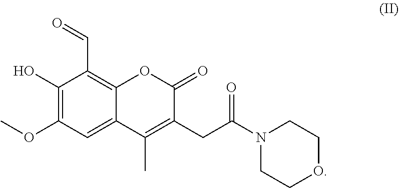 [SMILES: COc1cc2c(C)c(CC(=O)N3CCOCC3)c(=O)oc2c(C=O)c1O]

23. The method according to claim 19, wherein the cancer therapeutic agent is selected from the group consisting of cytotoxic chemotherapeutic agents; antimetabolites; antimitotic agents; alkylating agents; DNA damaging agents; antitumor antibiotics; platinum coordination complexes; proteasome inhibitors; HSP90 inhibitors; hormones and hormone analogs; aromatase inhibitors; fibrinolytic agents; antimigratory agents; antisecretory agents; immunosuppressives; anti-angiogenic compounds and vascular endothelial growth factor inhibitors; fibroblast growth factor inhibitors; epidermal growth factor receptor inhibitors; antibodies; checkpoint inhibitors; cell cycle inhibitors and differentiation inducers; mTOR inhibitors; corticosteroids; growth factor signal transduction kinase inhibitors; mitochondrial dysfunction inducers; caspase activators; chromatin disruptors and DNA repair enzyme inhibitors; HDAC inhibitors; Bcr-Abl inhibitors; FMS-like tyrosine kinase 3 (Flt3) inhibitors; and preferably selected from the group consisting of lestaurtinib, nilotinib, sorafenib, dasatinib, gefitinib, temisirolimus, vatalinib, Torisel®, vorinostat, paclitaxel, gemcitabine, 17-AAG, Velcade®, tamoxifen, temozolomide; or selected from the group consisting of sorafenib, eribulin, cyclophosphamide, 5-fluorouracil, carboplatin, doxorubicin, anastrozole; more preferably selected from the group consisting of paclitaxel, Velcade®, tamoxifen and temozolomide; or selected from the group consisting of sorafenib, eribulin, cyclophosphamide, 5-fluorouracil, carboplatin, doxorubicin.

24. The method according to claim 19, wherein the cancer therapeutic agent is used for treatment of cancerous tumor selected from the group consisting of liver cancer, triple negative breast cancer, estrogen positive breast cancer, ovarian carcinoma, pancreatic cancer, head and neck cancer, non-small cell lung cancer, glioblastoma, for example glioblastoma multiforme, and multiple myeloma.

25. The method according to claim 19, wherein
the cancer therapeutic agent is sorafenib; and
the cancer is liver tumor, preferably hepatocellular carcinoma.

26. The method according to claim 19, wherein
the cancer therapeutic agent is selected from
(i) microtubule disruptor, wherein said microtubule disruptor is selected from taxane and eribulin, and said taxane is selected from paclitaxel, docetaxel, cabazitaxel and albumin-bound paclitaxel, preferably paclitaxel and docetaxel, more preferably paclitaxel;
(ii) cyclophosphamide;
(iii) 5-fluorouracil;
(iv) carboplatin;
(v) doxorubicin; and
the cancer is breast cancer, preferably triple negative breast cancer,
or
the cancer therapeutic agent is selected from
(i) microtubule disruptor, wherein said taxane is selected from paclitaxel, docetaxel, cabazitaxel and albumin-bound paclitaxel, preferably paclitaxel and docetaxel, more preferably paclitaxel;
(ii) aromatase inhibitor, wherein said aromatase inhibitor is selected from letrozole and anastrozole;
(iii) tamoxifen; and
the cancer is breast cancer, preferably estrogen positive breast cancer, more preferably Her2 negative and estrogen positive metastatic breast cancer;
or
the cancer therapeutic agent is selected from taxane, wherein said taxane is selected from paclitaxel, docetaxel, cabazitaxel and albumin-bound paclitaxel, preferably paclitaxel and docetaxel, more preferably paclitaxel; and
the cancer is esophagus cancer (preferably esophageal squamous cell cancer), ovarian cancer, lung cancer (preferably non-small cell lung cancer), or glioblastoma.

27.-28. (canceled)

29. A method for treating cancerous tumors, comprising administering a subject in need thereof an effective amount of formula (I) or a pharmaceutically acceptable salt thereof: 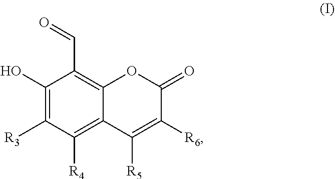
wherein
R₃ and R₄ are independently hydrogen or C₁₋₆ alkoxyl, which is optionally substituted with one or more substituents selected from the group consisting of (1) C₁-C₆ hydrocarbon chain containing N or O atom, and (2) C₃₋₁₀ cycloalkyl, which optionally contains 1 or 2 heteroatoms independently selected from the group consisting of N, O, and S;
R₅ is hydrogen, C₁₋₆ alkyl, C₁₋₆ alkoxyl, or C₁₋₆ alkylamino;
R₆ is C₁₋₆ alkyl, which is substituted with 1, 2 or 3 substituents independently selected from the group consisting of C₁₋₆ alkoxyl, C₁₋₆ hydroxylalkyl, C₁₋₆ alkoxyl C₁₋₆alkyl, 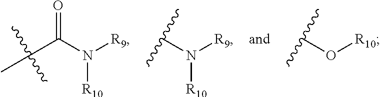
R₉ and R₁₀ are independently hydrogen; C₁₋₆ alkyl; C₁₋₆ alkoxyl C₁₋₆ alkyl; perfluoro C₁₋₆alkoxyl C₁₋₆alkyl; or
R₉ and R₁₀ together with the nitrogen atom to which they are attached form a heterocycle containing 1, 2, 3, or 4 heteroatoms independently selected from the group consisting of N, O, and S, and the heterocycle is optionally substituted with 1, 2, or 3 substituents independently selected from the group consisting of C₁₋₆ alkyl, C₁₋₆ alkylamino, C₁₋₆ alkoxyl;
and one or more additional cancer therapeutic agents.

30. The method according to claim 29, wherein the cancer therapeutic agent has at least one of the following features:
(1) inducing ER stress;
(2) inducing or up-regulating IRE-1α expression;
(3) inducing or up-regulating XBP1 splicing; and
(4) being less effective when IRE-1α is expressed.

31. The method according to claim 29, wherein the compound of formula (I) or the pharmaceutically acceptable salt thereof and one or more additional cancer therapeutic agents are administered simultaneously, separately or sequentially.

32. The method according to claim 29, wherein the compound of formula (I) has formula (II)

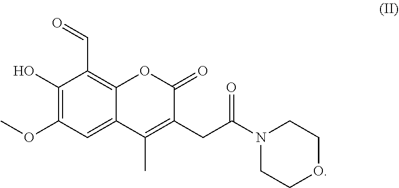 [SMILES: COc1cc2c(C)c(CC(=O)N3CCOCC3)c(=O)oc2c(C=O)c1O]

33. The method according to claim 29, wherein the cancer therapeutic agent is selected from the group consisting of cytotoxic chemotherapeutic agents; antimetabolites; antimitotic agents; alkylating agents; DNA damaging agents; antitumor antibiotics; platinum coordination complexes; proteasome inhibitors; HSP90 inhibitors; hormones and hormone analogs; aromatase inhibitors; fibrinolytic agents; antimigratory agents; antisecretory agents; immunosuppressives; anti-angiogenic compounds and vascular endothelial growth factor inhibitors; fibroblast growth factor inhibitors; epidermal growth factor receptor inhibitors; antibodies; checkpoint inhibitors; cell cycle inhibitors and differentiation inducers; mTOR inhibitors; corticosteroids; growth factor signal transduction kinase inhibitors; mitochondrial dysfunction inducers; caspase activators; chromatin disruptors and DNA repair enzyme inhibitors; HDAC inhibitors; Bcr-Abl inhibitors; FMS-like tyrosine kinase 3 (Flt3) inhibitors; and preferably selected from the group consisting of lestaurtinib, nilotinib, sorafenib, dasatinib, gefitinib, temisirolimus, vatalinib, Torisel®, vorinostat, paclitaxel, gemcitabine, 17-AAG, Velcade®, tamoxifen, temozolomide; or selected from the group consisting of sorafenib, eribulin, cyclophosphamide, 5-fluorouracil, carboplatin, doxorubicin, anastrozole; more preferably selected from the group consisting of paclitaxel, Velcade®, tamoxifen and temozolomide; or selected from the group consisting of sorafenib, eribulin, cyclophosphamide, 5-fluorouracil, carboplatin, doxorubicin.

34. The method according to claim 29, wherein the cancerous tumor is selected from the group consisting of liver cancer, triple negative breast cancer, estrogen positive breast cancer, ovarian carcinoma, pancreatic cancer, head and neck cancer, non-small cell lung cancer, glioblastoma, for example glioblastoma multiforme and multiple myeloma.

35. The method according to claim 29, wherein
the cancerous tumor is liver tumor, preferably hepatocellular carcinoma; and
the additional cancer therapeutic agent is sorafenib.

36. The method according to claim 29, wherein
the cancerous tumor is breast cancer, preferably triple negative breast cancer; and
the additional cancer therapeutic agent is selected from
(i) microtubule disruptor, wherein said microtubule disruptor is selected from taxane and eribulin, and said taxane is selected from paclitaxel, docetaxel, cabazitaxel and albumin-bound paclitaxel, preferably paclitaxel and docetaxel, more preferably paclitaxel;
(ii) cyclophosphamide;
(iii) 5-fluorouracil;
(iv) carboplatin;
(v) doxorubicin;
or
the cancerous tumor is breast cancer, preferably estrogen positive breast cancer, more preferably Her2 negative and estrogen positive metastatic breast cancer; and
the additional cancer therapeutic agent is selected from
(i) microtubule disruptor, wherein said taxane is selected from paclitaxel, docetaxel, cabazitaxel and albumin-bound paclitaxel, preferably paclitaxel and docetaxel, more preferably paclitaxel;
(ii) aromatase inhibitor, wherein said aromatase inhibitor is selected from letrozole and anastrozole;
(iii) tamoxifen;
or
the cancerous tumor is esophagus cancer (preferably esophageal squamous cell cancer), ovarian cancer, lung cancer (preferably non-small cell lung cancer), or glioblastoma; and
the additional cancer therapeutic agent is taxane, wherein said taxane is selected from paclitaxel, docetaxel, cabazitaxel and albumin-bound paclitaxel, preferably paclitaxel and docetaxel, more preferably paclitaxel.

37.-38. (canceled)


SEQUENCE LISTING (15 sequences)

SEQ ID NO: 1 - DNA, 22 bp, Artificial Sequence
  primer Human XBP1 Forward
ggaagccaaggggaatgaagtg

SEQ ID NO: 2 - DNA, 22 bp, Artificial Sequence
  primer Human XBP1 Reverse
ggagatgttctggaggggtgac

SEQ ID NO: 3 - DNA, 22 bp, Artificial Sequence
  primer GAPDH Forward
atcgtggaaggactcatgacca

SEQ ID NO: 4 - DNA, 22 bp, Artificial Sequence
  primer GAPDH Reverse
agggatgatgttctggagagcc

SEQ ID NO: 5 - DNA, 24 bp, Artificial Sequence
  probe for Human Unspliced XBP1
cacgtagtctgagtgctgcggact

SEQ ID NO: 6 - DNA, 19 bp, Artificial Sequence
  probe for Human Spliced XBP1
cctgcacctgctgcggact

SEQ ID NO: 7 - DNA, 22 bp, Artificial Sequence
  probe for GAPDH
tccatgccatcactgccaccca

SEQ ID NO: 8 - DNA, 20 bp, Artificial Sequence
  Primer for human XBP-1 forward
cctggttgctgaagaggagg

SEQ ID NO: 9 - DNA, 21 bp, Artificial Sequence
  primer for human XBP-1 reverse
ccatggggagatgttctggag

SEQ ID NO: 10 - DNA, 20 bp, Artificial Sequence
  primer for mouse forward
acacgcttgggaatggacac

SEQ ID NO: 11 - DNA, 20 bp, Artificial Sequence
  primer for mouse reverse
ccatgggaagatgttctggg

SEQ ID NO: 12 - DNA, 18 bp, Artificial Sequence
  mXBP-1 458
gaggccaaggggagtgga

SEQ ID NO: 13 - DNA, 24 bp, Artificial Sequence
  mXBP-1 572
agatgttctggggaggtgacaact

SEQ ID NO: 14 - DNA, 20 bp, Artificial Sequence
  mXBP-1 524
cacatagtctgagtgtgctg

SEQ ID NO: 15 - DNA, 19 bp, Artificial Sequence
  mXBP-1 580
cctgcacctgctgcggact